(19) United States
(12) Patent Application Publication
(10) Pub. No.: US 2026/0325214 A1
(43) Pub. Date: Oct. 22, 2026

(54) SYSTEM AND METHOD FOR TRANSSEPTAL PUNCTURE SITE SELECTION

(71) Applicant: Anumana, Inc., Cambridge, MA (US)

(72) Inventors: Suthirth Vaidya Subramany, Bengaluru (IN); Animesh Agarwal, San Mateo, CA (US)

(73) Assignee: Anumana, Inc., Cambridge, MA (US)

(21) Appl. No.: 19/185,745

(22) Filed: Apr. 22, 2025

(51) Int. Cl. A61B 34/10; A61B 17/00; A61B 90/00

(52) U.S. Cl. CPC A61B 34/10; A61B 17/00234; A61B 90/37; A61B 2034/105; A61B 2034/107; A61B 2090/3782

(57) ABSTRACT

A system and method for transseptal puncture site selection are disclosed. The system includes a processor and a memory communicatively connected to the processor, the memory containing instructions configuring the processor to receive a 3D organ model pertaining to an organ of a subject, receive a treatment plan pertaining to the subject, wherein the treatment plan includes at least an area of interest, and a procedure type, generate treatment guidance as a function of the treatment plan, and generate an overlay for the 3D organ model as a function of a puncture site and an optimized access route.

### FIELD OF THE INVENTION

The present invention generally relates to the field of technology-guided medical procedures. In particular, the present invention is directed to a system and method for transseptal puncture site selection.

### BACKGROUND

Numerous cardiac interventions, including left atrial appendage closure, mitral valve repair, left-sided ablations, and left-sided lead placements, require access to the left side of the heart. The most common method to achieve this access is through a transseptal puncture, wherein a hole is created in the interatrial septum to transition from the right atrium to the left atrium. Given the risks associated with left atrial instrumentation, such as thrombus formation and embolization, it is critical to optimize the puncture location to facilitate the specific procedure being performed.

### SUMMARY OF THE DISCLOSURE

In some aspects, the techniques described herein relate to a system for transseptal puncture site selection, including a processor and a memory communicatively connected to the processor, the memory containing instructions configuring the processor to receive a 3D organ model pertaining to an organ of a subject, receive a treatment plan pertaining to the subject, wherein the treatment plan includes at least an area of interest, and a procedure type, generate treatment guidance as a function of the treatment plan, wherein generating the treatment guidance includes determining an optimized access route of the treatment guidance to the area of interest, wherein the optimized access route is optimized for the procedure type, and locating a puncture site of the treatment guidance as a function of the treatment plan, wherein locating the puncture site includes determining the puncture site using a puncture machine-learning model, wherein the puncture machine-learning model is configured to receive the treatment plan as input and output a puncture site; and generate an overlay for the 3D organ model as a function of the puncture site and the optimized access route.

In some aspects, the techniques described herein relate to a method for transseptal puncture site selection, the method including receiving, by at least a processor, a 3D organ model pertaining to an organ of a subject, receiving, by the at least a processor, a treatment plan pertaining to the subject, wherein the treatment plan includes: at least an area of interest; and a procedure type, generating, by the at least a processor, treatment guidance as a function of the treatment plan, wherein generating the treatment guidance includes determining an optimized access route of the treatment guidance to the area of interest, wherein the optimized access route is optimized for the procedure type, and locating a puncture site of the treatment guidance as a function of the treatment plan, wherein locating the puncture site includes determining the puncture site using a puncture machine-learning model, wherein the puncture machine-learning model is configured to receive the treatment plan as input and output a puncture site, and generating, by the at least a processor, an overlay for the 3D organ model as a function of the puncture site and the optimized access route.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 illustrates a block diagram of an exemplary system for transseptal puncture site selection;

FIG. 2A-B illustrate exemplary embodiments of a graphical visualization;

FIG. 3 is an illustration of a downstream device displaying a color scale associated with a score during an ablation procedure;

FIG. 4 illustrates an exemplary embodiment of a three-dimensional (3D) voxel occupancy representation;

FIG. 5 illustrates a schematic diagram of an exemplary transesophageal echocardiogram;

FIG. 6 illustrates a block diagram of an exemplary embodiment of a machine-learning model;

FIG. 7 illustrates a schematic diagram of an exemplary embodiment of a neural network;

FIG. 8 illustrates a schematic diagram of an exemplary embodiment of a neural network node;

FIG. 9 illustrates a flow diagram of an exemplary method for transseptal puncture site selection; and

FIG. 10 illustrates a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for transseptal puncture site selection. In one or more embodiments, aspects of the present disclosure may eliminate unnecessary catheter manipulations, preventing cases where standard puncture sites lead to lengthy adjustments. In one or more embodiments, aspects of the present disclosure may reduce the risk of complications such as thrombus formation and embolization, and improved procedural outcomes by ensuring consistent and optimized device placement, improving success rates for interventions. In one or more embodiments, a system such as any system as described in this disclosure may minimize fluoroscopy dependence by integrating real-time ultrasound visualization. In one or more embodiments, the system has the potential to reduce radiation exposure to both the patient and the physician. In one or more embodiments, aspects of the present disclosure can be incorporated into existing imaging platforms used in catheterization labs. In one or more embodiments, a combination of machine-learning and geometric modeling may be used to refine site selection and path prediction based on procedural outcomes. In one or more embodiments, aspects of the present disclosure may provide a novel, data-driven approach to transseptal puncture planning and execution, significantly improving procedural efficiency and patient safety. By leveraging 3D ultrasound imaging and computational algorithms, the system enables precise, patient-specific transseptal access, paving the way for safer and more efficient left-heart interventions.

In some aspects, the techniques described herein relate to a system for transseptal puncture site selection, including a processor and a memory communicatively connected to the processor, the memory containing instructions configuring the processor to receive a 3D organ model pertaining to an organ of a subject, receive a treatment plan pertaining to the subject, wherein the treatment plan includes at least an area of interest, and a procedure type, generate treatment guidance as a function of the treatment plan, wherein generating the treatment guidance includes determining an optimized access route of the treatment guidance to the area of interest, wherein the optimized access route is optimized for the procedure type, and locating a puncture site of the treatment guidance as a function of the treatment plan, wherein locating the puncture site includes determining the puncture site using a puncture machine-learning model, wherein the puncture machine-learning model is configured to receive the treatment plan as input and output a puncture site; and generate an overlay for the 3D organ model as a function of the puncture site and the optimized access route.

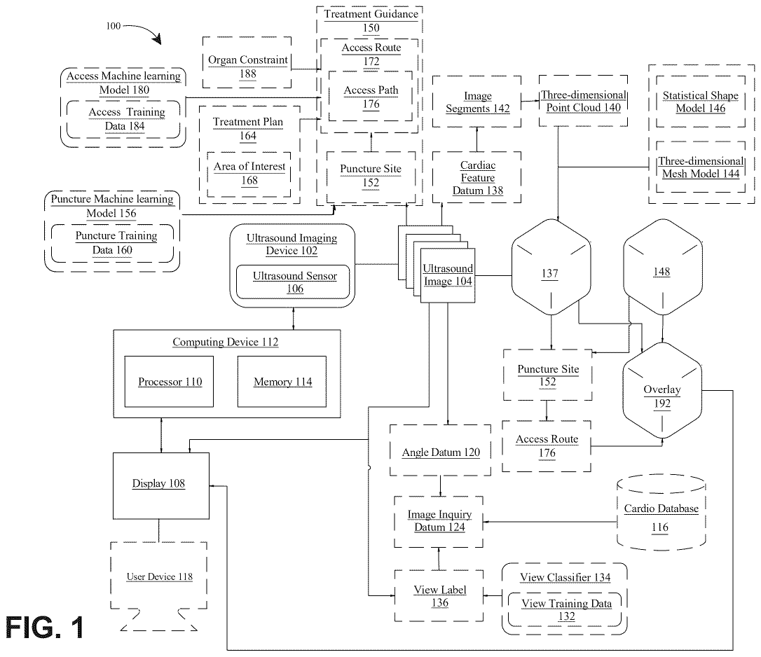

Referring now to FIG. 1, an exemplary system 100 for puncture site selection is described. In one or more embodiments, system 100 may include an ultrasonic imaging device. As used herein, an “ultrasonic imaging device” is a device configured to collect ultrasonic images. Non-limiting examples of ultrasonic images and/or imaging techniques include intracardiac echo (ICE) images, transthoracic echocardiograms (TTE), transesophageal echocardiograms (TEE), and point of care ultrasound (POCUS). In one or more embodiments, ultrasonic imaging device may be configured to capture a plurality of ultrasonic images. As used herein, an “ultrasonic image” is an image generated as a function of a reflection of a sound wave off of a structure. In some embodiments, plurality of ultrasonic images may include a TEE image. In some embodiments, a TEE image may include a 2D TEE. In some embodiments, a TEE image may include a 3D TEE. In some embodiments, plurality of ultrasonic images may include an ICE image. In some embodiments, a set of ultrasonic images of the patient's organ may include an image selected from the list consisting of a transesophageal echocardiogram image, a transthoracic echocardiogram image, and a point-of-care ultrasound image. An ultrasonic image of plurality of ultrasonic images may depict an organ and/or tissue of a subject. An ultrasonic image of plurality of ultrasonic images may depict a heart, lung, spleen, liver, kidney, muscle, skeleton, intestine, stomach, vein, and/or artery. In some embodiments, an ultrasonic image may depict a heart, and/or a left atrium, left atrial appendage, left ventricle, right ventricle, and/or a right atrium of a heart.

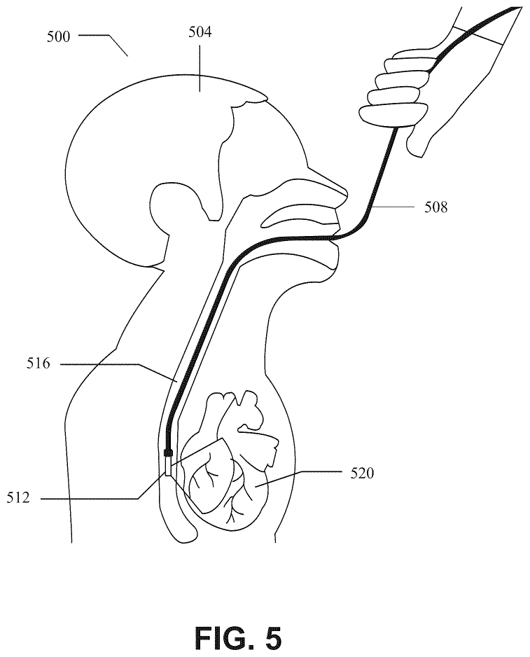

Still referring to FIG. 1, in some embodiments, an ultrasonic image may include a transesophageal echocardiogram (TEE) image. In some embodiments, a TEE image may include a view of an ostial diameter and/or length. In some embodiments, plurality of ultrasonic images may include ultrasonic images captured at the same position, and different orientations with respect to a subject's heart. In some embodiments, a TEE image may include a view of an ostial diameter and/or length from one or more angles on a mid-esophageal view. As examples, a TEE image may include a view of a subject's heart and/or an ostial diameter and/or length from 1, 2, 3, 4, 5, 6, 7, 8, 9, or 10 different angles on a mid-esophageal view. In a non-limiting example, a TEE image may include a view of a subject's heart and/or an ostial diameter and/or length from 4 different angles (0°, 45°, 90°, 135°) on a mid-esophageal view. In some embodiments, angles of images may describe a rotational position of an ultrasound transducer relative to a subject's heart. In additional non-limiting examples, a TEE image may include a view of a subject's heart and/or an ostial diameter and/or length from 0° (which may provide a four chamber view), 45-60° (which may provide a two chamber view and in some embodiments may be used to identify a thrombus), 90° (which may provide a long-axis view), 120-135° (which may provide a long-axis view of a left atrial appendage) and/or 135-160° (which may provide an aortic valve long axis view) on a mid-esophageal view. In some embodiments, an ostial diameter and/or length includes an ostial diameter and/or length of a left atrial appendage (LA A) of a heart and/or another location at which a cardiac implant may be placed. In some embodiments, TEE image may include a view of an ostial diameter and/or length from 4 different angles, such as, as a non-limiting example, 0°, 45°, 90°, and 135°. TEE imaging is discussed further with respect to FIG. 5.

Still referring to FIG. 1, in some embodiments, an ultrasonic image may include an intracardiac echocardiography (ICE) image. As used herein, an “ICE image” is an ultrasound image obtained from within the heart's chambers or blood vessels. In some cases, ICE images may be captured using a specialized catheter equipped with an ultrasound transducer that is inserted into the body and guided to the heart of subject. In an embodiment, an ultrasound image may provide a detailed and real-time visualization of cardiac anatomy. ICE images may also include internal structures, functions, and blood flow patterns of the heart of a subject. Plurality of ultrasonic images may be related in terms of content, time of capture, sequence, or any other relevant parameters described herein. In a non-limiting example, each image of plurality of ultrasonic images may represent a particular view, angle, or perspective of an object, subject, or scene, and may be in two-dimensional (2D) or 3D format. Images of plurality of ultrasonic images may include, without limitation, any two-dimensional or three-dimensional images of any anatomy or anatomical structure, including without limitation, images of any internal organ, tissue including muscular, connective tissue, epithelial tissue, and/or nervous tissue, bone, and/or any other element that may be imaged within a human and/or animal body. In one or more embodiments, system 100 includes at least an intracardiac echocardiography system including at least an ultrasound sensor configured to be inserted into a vein of the subject and capture one or more ultrasound images and/or ICE images of a heart of the subject. In one or more embodiments, intracardiac echocardiography system may be configured to capture plurality of ultrasound images and/or ICE images.

Still referring to FIG. 1, in a non-limiting example, structures of a heart of a subject which may be imaged may include chambers (e.g., four chambers including left and right atria and left and right ventricles), valves (i.e., the structures that regulate blood flow between chambers and vessels, including mitral, tricuspid, aortic, and pulmonary valves), vessels (e.g., aorta, pulmonary arteries and veins, and coronary arteries), conduction system (i.e., a network of specialized cells that control the heart's electrical activity and rhythm), muscular and connective tissues (e.g., heart's muscular walls, septa, any other connective tissues that provide structural integrity and enable contraction), LAA and other appendages, pathological features (e.g., any abnormalities, defects, and/or the like), and/or other components of a heart.

Still referring to FIG. 1, as used in this disclosure, a “subject” refers to an individual organism. In an embodiment, subject may include a human, such as a human in need of a cardiac implant. A cardiac implant may include a left atrial appendage occlusion (LAAO) device. A cardiac implant may include a watchman device. A watchman device may fit in the left atrium of a subject and form a barrier against blood clots to reduce the risk of stroke. Different patients may receive different size devices, and cardiac measurements made as described herein may be used to determine which size device to give to a particular subject.

Still referring to FIG. 1, in an embodiment, each ultrasonic image of plurality of ultrasonic images may include a particular view of subject's heart's chambers, valves, vessels, and/or the like. In a non-limiting example, plurality of ultrasonic images may include multiple views e.g., different angles and perspectives of a subject's heart. In another embodiment, plurality of ultrasonic images may be arranged in a temporal sequence. In a non-limiting example, plurality of ultrasonic images may include a series of images captured over time, allowing for an observation of dynamic cardiac functions such as beating, blood flow, and/or the like. In some cases, each ultrasonic image of plurality of ultrasonic images may include a corresponding timestamp, wherein the timestamp may include an indicator showing a date and time of when the corresponding ultrasonic image was taken.

With continued reference to FIG. 1, in one or more embodiments, system 100 may include at least a transesophageal echocardiogram (TEE) system. For the purposes of this disclosure, a “transesophageal echocardiography (TEE) system” is a system designed to perform transesophageal echocardiography. For the purposes of this disclosure, “transesophageal echocardiography” is a medical imaging technique in which an ultrasound transducer is inserted into the esophagus to produce images of the heart and surrounding structures. In one or more embodiments, TEE system may include a combination of specialized hardware and software designed to facilitate transesophageal echocardiography by positioning an ultrasound probe (e.g., ultrasound transducer) within esophagus of a patient, close to the heart of the patient. As the esophagus is proximate to the heart, a TEE system can detect ultrasound image 104 of cardiac tissue of a patient. In one or more embodiments, ultrasound imaging device 102 may include a TEE probe (endoscope). The ultrasound probe is a long, flexible device equipped with an ultrasound transducer (ultrasound sensor 106) at its tip. Ultrasound transducer can emit high-frequency sound waves and capture the echoes reflected from cardiac structures to produce detailed images (ultrasound image 104). In a non-limiting example, ultrasound probe may be inserted into the patient's esophagus, where the ultrasound probe may be connected to an ultrasound machine, which processes signals from ultrasound transducer to generate images of the heart. In one or more embodiments, ultrasound imaging device 102 may be communicatively connected to at least a display 108. The display disclosed herein is further described in detail below. Additional disclosure related to ultrasound imaging device 102 is further described in detail below.

With continued reference to FIG. 1, ultrasound imaging device 102 may include at least an ultrasound sensor 106 configured to be located within an esophagus of a patient and detect at least an ultrasound image 104. In one or more embodiments, ultrasound sensor may be configured to capture one or more ultrasound images of subject. For the purposes of this disclosure, an “ultrasound sensor” is a device that uses ultrasonic waves to detect the presence and distance of objects. In a non-limiting example, ultrasound sensor 106 may measure the distance to an object using ultrasonic sound waves. For the purposes of this disclosure, a “sensor” is a device that produces an output signal for the purpose of sensing a physical phenomenon. For example, and without limitation, sensor may transduce a detected phenomenon, such as without limitation, temperature, voltage, current, pressure, speed, motion, light, moisture, sound waves, and the like, into a sensed signal. Sensor may output the sensed signal. Sensor may include any computing device as described in the entirety of this disclosure and configured to convert and/or translate a plurality of signals detected into electrical signals for further analysis and/or manipulation. Electrical signals may include analog signals, digital signals, periodic or aperiodic signal, step signals, unit impulse signal, unit ramp signal, unit parabolic signal, signum function, exponential signal, rectangular signal, triangular signal, sinusoidal signal, sine function, or pulse width modulated signal. A ny datum captured by sensor may include circuitry, computing devices, electronic components or a combination thereof that translates into at least an electronic signal configured to be transmitted to another electronic component. In a non-limiting embodiment, sensor may include a plurality of sensors included in a sensor suite. In one or more embodiments, and without limitation, sensor may include a plurality of sensors. Sensor may include an ultrasound sensor 106.

With continued reference to FIG. 1, in one or more embodiments, ultrasound sensor 106 may include an electrode. For the purposes of this disclosure, an “electrode” is a conductive material or element that facilitates the transmission and reception of electrical signals associated with ultrasound waves. In a non-limiting example, electrode may detect and record electrical activity; for instance, but not limited to, the heart's electrical signals. For example, and without limitation, electrode may generate ultrasonic sound waves, from which ultrasound sensor 106 receives the ultrasonic waves and transmit ultrasound image 104 related to the ultrasonic waves to processor 110. In one or more embodiments, ultrasound sensor 106 may include a transducer. For the purposes of this disclosure, a “transducer” is a component of an ultrasound sensor that converts one form of energy into another. In a non-limiting example, transducer may operate on a principle of piezoelectricity, where piezoelectric material can convert electrical energy into mechanical vibration (i.e., ultrasonic waves) and vice versa. In one or more embodiments, ultrasound sensor 106 may include a transceiver. For the purposes of this disclosure, a “transceiver” is a combined unit of a transmitter and a receiver. In a non-limiting example, transceiver may transmit ultrasonic waves and receive echoes.

With continued reference to FIG. 1, for the purposes of this disclosure, “ultrasound image” is a visual representation generated by reflection of high-frequency sound waves off internal body structures. In a non-limiting example, ultrasound image 104 may include visual representation of a heart examined through esophagus and/or veins of subject. As a non-limiting example, ultrasound image 104 may include distance between sensor and surrounding tissue or organs. In one or more embodiments, ultrasound sensor 106 may detect ultrasound image 104 in a plurality of angles. In a non-limiting example, ultrasound image 104 may include a plurality of distances between sensor and a heart in different angles. For example, and without limitation, when ultrasound sensor 106 moves around within an esophagus, ultrasound sensor 106 receives a plurality of distances between ultrasound sensor 106 and organ and generate ultrasound image 104 using the plurality of distances.

With continued reference to FIG. 1, in one or more embodiments, ultrasound sensor may be configured to capture an ultrasound image 104 of a cardiac tissue of a patient. “Cardiac tissue” as described in this disclosure refers to any portion of an individual's heart. For example, and without limitation, cardiac tissue may include heart valves, chambers, protective sacs and/or the like. In a non-limiting example, ultrasound image 104 may include an image of a heart before implantation of a cardiovascular device, during implantation of a cardiovascular device and/or after implantation of a cardiovascular device. For example, without limitation, ultrasound image 104 may include an image of a valve of a heart that may not be working properly. In one or more embodiments, ultrasound sensor may be configured to capture the internal surroundings of a patient's heart.

With continued reference to FIG. 1, system 100 includes a computing device 112. Computing device 112 includes a processor 110 communicatively connected to a memory 114. As used in this disclosure, “communicatively connected” means connected by way of a connection, attachment or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device 112. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

With continued reference to FIG. 1, in one or more embodiments, computing device 112 may include any computing device as described in this disclosure, including without limitation, a microcontroller, microprocessor, digital signal processor (DSP) and/or system on a chip (SoC) as described in this disclosure. Computing device 112 may include, be included in, and/or communicate with a mobile device such as a mobile telephone or smartphone. Computing device 112 may include a single computing device operating independently, or may include two or more computing devices operating in concert, in parallel, sequentially or the like; two or more computing devices may be included together in a single computing device or in two or more computing devices. Computing device 112 may interface or communicate with one or more additional devices as described below in further detail via a network interface device. Network interface device may be utilized for connecting computing device 112 to one or more of a variety of networks, and one or more devices. Examples of a network interface device include, but are not limited to, a network interface card (e.g., a mobile network interface card, an LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software.) may be communicated to and/or from a computer and/or a computing device 112. Computing device 112 may include but is not limited to, for example, a computing device or cluster of computing devices in a first location and a second computing device or cluster of computing devices in a second location. Computing device 112 may include one or more computing devices dedicated to data storage, security, distribution of traffic for load balancing, and the like. Computing device 112 may distribute one or more computing tasks as described below across a plurality of computing devices of computing device, which may operate in parallel, in series, redundantly, or in any other manner used for distribution of tasks or memory between computing devices. Computing device 112 may be implemented, as a non-limiting example, using a “shared nothing” architecture.

With continued reference to FIG. 1, in one or more embodiments, computing device 112 may be designed and/or configured to perform any method, method step, or sequence of method steps in any embodiment described in this disclosure, in any order and with any degree of repetition. For instance, computing device 112 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 112 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, memory 114 contains instructions configuring processor 110 to receive at least an ultrasound image 104. In one or more embodiments, processor 110 may receive ultrasound image 104 from TEE system, intracardiac echocardiography system and/or ultrasound imaging device. In one or more embodiments, processor 110 may receive ultrasound image 104 from a cardio database 116. In one or more embodiments, system 100 may include a cardio database 116. As used in this disclosure “cardio database” is a data structure configured to store data associated with one or more patient's hearts. In one or more embodiments, cardio database 116 may include inputted or calculated information and datum related to a patient's heart such as chambers, blood vessels and/or the like. In one or more embodiments, a datum history may be stored in cardio database 116. As a non-limiting example, the datum history may include real-time and/or previous inputted data related to the patient's heart. As a non-limiting example, cardio database 116 may include instructions from a user, who may be an expert user, a past user in embodiments disclosed herein, or the like, where the instructions may include examples of the data related to a patient's heart.

With continued reference to FIG. 1, in one or more embodiments, processor 110 may be communicatively connected with cardio database 116. For example, and without limitation, in one or more embodiments, cardio database 116 may be local to processor 110. In another example, and without limitation, cardio database 116 may be remote to processor 110 and communicative with processor 110 by way of one or more networks. The network may include, but is not limited to, a cloud network, a mesh network, and the like. By way of example, a “cloud-based” system can refer to a system which includes software and/or data which is stored, managed, and/or processed on a network of remote servers hosted in the “cloud,” e.g., via the Internet, rather than on local severs or personal computers. A “mesh network” as used in this disclosure is a local network topology in which the infrastructure processor connect directly, dynamically, and non-hierarchically to as many other computing devices as possible. A “network topology” as used in this disclosure is an arrangement of elements of a communication network. The network may use an immutable sequential listing to securely store cardio database 116. An “immutable sequential listing,” as used in this disclosure, is a data structure that places data entries in a fixed sequential arrangement, such as a temporal sequence of entries and/or blocks thereof, where the sequential arrangement, once established, cannot be altered or reordered. An immutable sequential listing may be, include and/or implement an immutable ledger, where data entries that have been posted to the immutable sequential listing cannot be altered.

With continued reference to FIG. 1, in one or more embodiments, cardio database 116 may be implemented, without limitation, as a relational database, a key-value retrieval database such as a NOSQL database, or any other format or structure for use as a database that a person skilled in the art would recognize as suitable upon review of the entirety of this disclosure. Database may alternatively or additionally be implemented using a distributed data storage protocol and/or data structure, such as a distributed hash table or the like. Database May include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure.

With continued reference to FIG. 1, in one or more embodiments, processor 110 may receive ultrasound image 104 from a user device 118. For the purposes of this disclosure, a “user device” is any device a user use to input data. For the purposes of this disclosure, a “user” is an individual or entity that uses a system. As a non-limiting example, a user may include a surgeon, doctor, medical professional, and the like. As a non-limiting example, user device 118 may include a laptop, desktop, tablet, mobile phone, smart phone, smart watch, kiosk, screen, smart headset, or the like. In one or more embodiments, user device 118 may include an interface configured to receive inputs from user. In one or more embodiments, user may manually input any data into computing device 112 using user device 118. In one or more embodiments, user may have the capability to process, store or transmit any information independently.

With continued reference to FIG. 1, in one or more embodiments, receiving at least an ultrasound image 104 may include extracting at least an angle datum 120 from at least an ultrasound image 104 using optical character recognition. For the purposes of this disclosure, an “angle datum” is a data element indicating a value that represents the angular orientation of an ultrasound sensor. In one or more embodiments, angle datum 120 may be angular orientation of an ultrasound sensor 106 relative to a reference axis or plane (e.g., the anatomical position of a heart). As a non-limiting example, angle datum 120 may include probe's (e.g., ultrasound sensor 106) imaging angle, such as 0°, 45°, 90°, or 135°, which may determine the plane of the ultrasound slice captured during imaging.

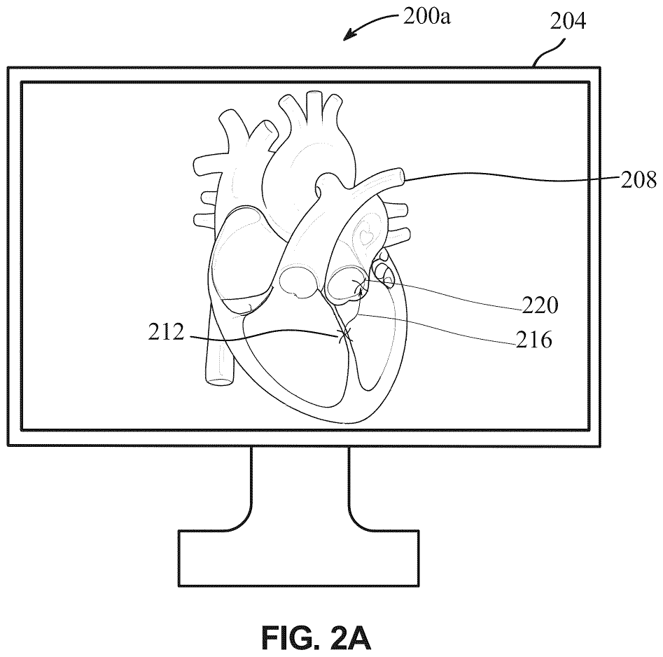

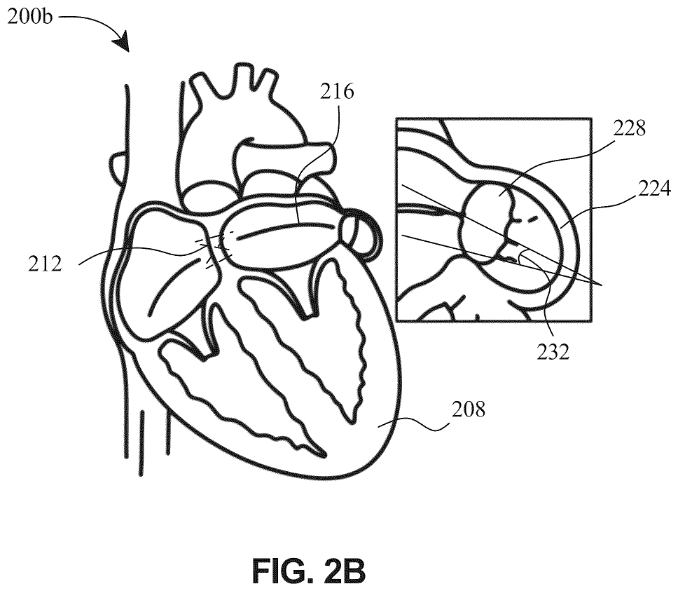

With continued reference to FIG. 1, ultrasound image 104 may be received through a TEE procedure. In one or more embodiments, ultrasound image may be received from ultrasound imaging device, TEE system and/or ultrasound sensor 106 during a TEE procedure. TEE procedure is described in further detail below in reference to at least FIG. 2. In one or more embodiments, ultrasound image may include TEE echocardiogram data as described in reference to at least FIG. 2.

With continued reference to FIG. 1, memory 114 contains instructions configuring processor 110 to receive at least a 3D organ model 137 representative of a patient's heart as a function of at least an ultrasound image 104. In one or more embodiments, processor 110 is configured to receive 3D organ model 137 pertaining to subject. For the purposes of this disclosure, a “three-dimensional cardiac model” is a three-dimensional representation of a patient's heart and/or surrounding structures. In some embodiments, 3D organ model 137 may include peripheral vasculature. “Peripheral vasculature,” for the purposes of this disclosure, is a structure or structures of blood vessels surrounding or in the immediate vicinity of the heart. In a non-limiting example, 3D organ model 137 may include a 3D voxel occupancy representation (VOR). As used in this disclosure, a “3D voxel occupancy representation (VOR)” of an anatomical object is a 3D digital representation of a spatial structure of the anatomical object, wherein the representation is composed of a plurality of discrete volumetric elements known as voxels. A “voxel,” for the purpose of this disclosure, is a 3D equivalent of a pixel in 2D imaging. While a pixel represents a point in a 2D image and may include properties such as color and/or brightness, a voxel may represent a volume in a 3D space and may include additional properties such density/occupancy as described below. In an embodiment, each voxel of plurality of voxels within 3D VOR may represent a specific portion of heart. In one or more embodiments, voxel may be a smallest distinguishable box-shaped part (i.e., 1 px·1 px·1 px) of a three-dimensional image. In one or more embodiments, each voxel of plurality of voxels within VOR may be represented as a cube or rectangular prism (although other shapes may be used in specialized applications). Each voxel may include a size that determines a resolution of the 3D image or model. In an embodiment, smaller voxels may provide higher resolution; however, it may require more computational resources (e.g., RAM) for processor 110 to process.

In an embodiment, and still referring to FIG. 1, each voxel of plurality of voxels within VOR may include one or more embedded values. As used herein, “embedded values” refers to specific numerical or categorical data associated with each voxel. In one or more embodiments, embedded values may represent various attributes or characteristics of the corresponding portion of heart that voxel represents. In a non-limiting example, embedded values may include density values, intensity values, texture information, or any other quantitative measures that provide insights into the underlying cardiac tissue. Such embedded values may be derived from set of ultrasonic images or other imaging modalities used to generate 3D organ model 137. In one or more embodiments, embedded values may be utilized, by processor 110, to differentiate between different types of cardiac tissues, such as myocardial tissue, blood vessels, or chambers. Embedded values may also facilitate the visualization of dynamic cardiac functions, for example, and without limitation, blood flow or heart beating by encoding temporal information such as timestamps within plurality of voxels.

In one or more embodiments, and still reference to FIG. 1, one or more embedded values, such as, without limitations, occupancy, or density, may be derived from ultrasound images described herein by processor 110. In a non-limiting example, determining occupancy status of each voxel of plurality of voxels may include converting set of ultrasonic images to a set of binary images and determining occupancy status of each voxel as a function of the structure of interest's binary value. In one or more embodiments, occupancy status may include a value representing the likelihood of occupancy of the corresponding heart tissue. In another non-limiting example, density may be calculated, by processor 110, for each voxel as a function of the echogenicity of one or more pixels on a given ultrasound image 104, wherein, the brightness of the given ultrasonic image may be analyzed since different tissues reflect ultrasound waves differently.

With continued reference to FIG. 1, generating 3D organ model 137 of heart may include generating a 3D array. In one or more embodiments, 3D organ model 137 may be generated as a function of angle datum 120, wherein orientation of ultrasound images may be determined using angle datum 120. In one or more embodiments, processor 110 may divide 3D space into a grid of plurality of voxels, each with specific x, y, and z coordinates as embedded values. Each element of 3D array may correspond to a voxel. In one or more embodiments, 3D array may allow for easy access and manipulation of plurality of voxels, enabling various analyses, visualizations, and transformations either described or not described herein. In a non-limiting example, embedded values may include a density of the tissue at a specific location of a patient's body derived from one or more ultrasonic images of ultrasound images.

Additionally, or alternatively, and still referring to FIG. 1, 3D organ model 137 of heart may include a 3D grid embedded values described herein of plurality of voxels (e.g., tissue density, blood flow velocity, echogenicity or acoustic properties, and any other biophysical properties). As used in this disclosure, a “3D grid” refers to a 3D data structure that divides a given volume (e.g., volume of a heart) into a plurality of discrete units called cells (i.e., volume elements). In an embodiment, each cell within 3D grid may be associated with a distinct voxel.

In yet another embodiment, and still referring to FIG. 1, cells may be continuous, meaning that one or more cells may represent one or more continuous regions of space rather than discreate, separate units. In a non-limiting example, instead of being uniform, mapped presence indicator and/or other embedded values may vary continuously across different cells or cell's volume. In such embodiment, processor 110 may use interpolation to estimate other (unknown) embedded values within a range based on existing values such as known embedded values at specific points, thereby allowing for smooth transitions between cells. Exemplary interpolation methods may include, without limitation, linear interpolation, cubic interpolation, and/or the like. For example, and without limitation, if the corners of a cell have known values interpolation can be used to estimate the values at any point within the cell based on those corner values.

In a non-limiting example, and still referring to FIG. 1, 3D organ model 137 of heart may include a 3D grid having a plurality of cells e.g., voxels, wherein each cell may contain a continuous range of values representing tissue density, blood flow velocity, or other properties (i.e., embedded values). Processor 110 may be configured to apply trilinear or tricubic interpolation to estimate tissue density within each cell based on presence indicator or other known values at the cell's boundaries, since tissue densities change gradually; Such 3D grid may provide a smooth, continuous representation of heat's internal structures, allowing for more nuanced analysis and visualization as described below. In a further embodiment, 3D grid with continuous cells may be additionally used in fluid dynamics simulations.

With continued reference to FIG. 1, in some case, embedded values may be mapped to 3D grid as a function of array masking, wherein specific array or grid may be selected to modify based on one or more pre-defined criteria. In a non-limiting example, processor 110 may generate a mask e.g., a binary array that defines which voxels or cells are affected. M ask may be used to select or modify specific voxels or cells based on certain attributes; for instance, without limitation, processor 110 may use mask to isolate the left atrium (LA) within the heart focusing the analysis on that specific region. Such mask may include a criterion defined by specific density thresholds that distinguish the LA's tissue (i.e., voxels representing LA in 3D grid) from surrounding structures (i.e., neighboring voxels). In one or more embodiments, such mask may further include a binary mask, wherein each voxel in the 3D gird may be assigned a first presence indicator such as 1 if the voxel meets the criteria for the LA and a second presence indicator such as 0 if it does not. In one or more embodiments, mask may be directly applied to 3D grid, selecting, or modifying voxels or cells, thereby enabling processor 110 to highlight, exclude, or otherwise manipulate specific parts of heart within 3D grid. Processor 110 may then perform an element-wise multiplication between 3D grid and the mask. Continuing from the previous non-limiting example, voxels corresponding to the LA (wherein the mask value is 1) may retain their original values, while other voxels (where the mask value is 0) may be set to 0 or other specific value (i.e., excluded or masked out).

With continued reference to FIG. 1, ultrasound image 104 may include a two-dimensional representation of a patient's heart. In one or more embodiments, two-dimensional representation may include two-dimensional images. In one or more embodiments, two-dimensional images may include a flat, planar representation containing rows and columns of pixel values, such as a photograph or a digital drawing. In one or more embodiments, ultrasound image may be created by converting ultrasound waves into a two-dimensional image. In one or more embodiments, brightness and shading within ultrasound image 104 may be determined based on the intensity of returning echoes. In one or more embodiments, intensity of returning echoes may depend on the density and type of tissue. In one or more embodiments, ultrasound imaging device 102 may be configured to receive ultrasound waves from ultrasound sensor and generate ultrasound images as result. In one or more embodiments, 3D organ model 137 includes a three-dimensional interpolation of the two-dimensional image of ultrasound image 104. A “three-dimensional interpolation,” as described in this disclosure refers to a process of using data from a two-dimensional image or from textual data to generate or estimate values in a three-dimensional space. For example, and without limitation, images may be used to construct a three-dimensional model of the image. In one or more embodiments, a three-dimensional model may include a length width and height, whereas a two-dimensional image and/or model may include a length and height. In one or more embodiments, processor 110 may use any process as described in this disclosure to generate 3D organ model 137, wherein 3D organ model 137 includes a three-dimensional interpolation of a two-dimensional image.

With continued reference to FIG. 1, in one or more embodiments, processor 110 may generate an image inquiry datum 124 as a function of the at least an angle datum 120 retrieved from Cardio database 116. For the purposes of this disclosure, an “image inquiry datum” is a data element that inquires additional ultrasound images. In one or more embodiments, image inquiry datum 124 may include a control signal that is transmitted to TEE system to obtain necessary additional ultrasound images. In a non-limiting example, if processor 110 determines ultrasound images in specific views or specific angles and/or a number of ultrasound images are missing, processor 110 may request additional ultrasound images from TEE system. In one or more embodiments, image inquiry datum 124 may include a notification for a user. In a non-limiting example, if processor 110 determines that ultrasound images in specific views or specific angles and/or a number of ultrasound images are missing based on missing portions of a heart, missing coordinates points, missing cardiac features datums and/or the like, processor 110 may generate a notification and may transmit to a user to operate TEE system to generate additional ultrasound images more and/or in the specific views or specific angles. In a non-limiting example, image inquiry datum 124 may be configured to query additional ultrasound images to a user of at least a TEE system through at least a display 108.

With continued reference to FIG. 1, in one or more embodiments, receiving at least an ultrasound image 104 may include generating view training data 132, wherein the view training data 132 may include exemplary ultrasound images correlated to exemplary view labels, training a view classifier 134 using the view training data 132, classifying the at least an ultrasound image 104 to at least a view label 136 using the trained view classifier 134 and generating an image inquiry datum 124 as a function of the view label 136. For the purposes of this disclosure, a “view label” is an identifier associated with an imaging perspective or orientation of a heart captured during a transesophageal echocardiography. As a non-limiting example, view label 136 may include mid-esophageal four-chamber view, mid-esophageal bicaval view, mid-esophageal long-axis view, mid-esophageal left atrial appendage view, transgastric short-axis view, transgastric two-chamber view, aortic valve short-axis view, and the like. In one or more embodiments, view label 136 may be retrieved from Cardio database 116. In one or more embodiments, user may manually input view label 136 of ultrasound images.

With continued reference to FIG. 1, in one or more embodiments, view training data 132 may be stored in Cardio database 116. For the purposes of this disclosure, “view training data” is data containing correlations that a machine-learning process may use to model relationships between ultrasound images and view labels. In one or more embodiments, view training data 132 may be received from one or more users, Cardio database 116, external computing devices, and/or previous iterations of processing. As a non-limiting example, view training data 132 may include instructions from a user, who may be an expert user, a past user in embodiments disclosed herein, or the like, which may be stored in memory and/or stored in Cardio database 116, where the instructions may include labeling of training examples. In one or more embodiments, view training data 132 may be updated iteratively on a feedback loop. As a non-limiting example, processor 110 may update view training data 132 iteratively through a feedback loop as a function of ultrasound image 104, angle datum 120, view label 136, or the like. In one or more embodiments, processor 110 may be configured to generate a view classifier 134. For the purposes of this disclosure, a “view classifier” is a machine-learning model as defined below, such as a data structure representing and/or using a mathematical model, neural net, or program generated by a machine learning algorithm known as a “classification algorithm,” as described in further detail below, that sorts ultrasound images into view labels. In a non-limiting example, generating view classifier 134 may include training, retraining, or fine-tuning view classifier 134 using view training data 132 or updated view training data 132. In one or more embodiments, processor 110 may be configured to determine view label 136 using view classifier 134 (i.e., trained or updated view classifier 134). In one or more embodiments, generating training data and training machine-learning models may be simultaneous.

With continued reference to FIG. 1, in one or more embodiments, 3D grid may include one or more cardiac feature datums extracted from ultrasound image 104 of heart. As used in this disclosure, “cardiac feature datums” are specific characteristics or attributes related to a heart. In one or more embodiments, cardiac feature datum 138 may include spatial arrangement, shape, size, or texture of a heart. In one or more embodiments, cardiac feature datums may include one or more embedded values described herein and their combinations thereof. In a non-limiting example, cardiac feature datum 138 may be represented numerically as a vector, a metric or other mathematical constructs that capture specific spatial characteristics. In one or more embodiments, cardiac feature datums may also be visualized as contours, surfaces, or other geometric representations. In an embodiment, cardiac feature datums may be extracted using edge detection, texture analysis, or other image processing techniques (e.g., cleaning and enhancing images, image segmentation, and/or the like). In another embodiment, one or more machine-learning models, such as convolutional neural networks (CNNs) as described in further detail below, may be used to extract complex cardiac feature datums. The cardiac feature datum 138 is further described below. In one or more embodiments, cardiac feature datum 138 may further include the size and/or placement of arteries, identification of various cardiovascular devices and/or the like. In one or more embodiments, cardiac feature datum 138 may indicate a particular portion of the heart, such as for example, a specific valve, a specific chamber, a specific cardiovascular device and/or the like.

Still referring to FIG. 1, as used in this disclosure, a “vector” is a data structure that represents one or more a quantitative values and/or measures of one or more cardiac feature datums. A vector may be represented as an n-tuple of values, where n is one or more values, as described in further detail below; a vector may alternatively or additionally be represented as an element of a vector space, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent, for instance as measured using cosine similarity as computed using a dot product of two vectors; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute/as derived using a Pythagorean norm:

$$l = \sqrt{{\sum }_{i = 0}^{n} {a}_{i}^{2}} ,$$

where aᵢ is attribute number i of the vector. Scaling and/or normalization may function to make vector comparison independent of absolute quantities of attributes, while preserving any dependency on similarity of attributes.

Still referring to FIG. 1, in a non-limiting example, one or more cardiac feature datums may include one or more shape features (i.e., characteristics related to the shape of specific cardiac structures), such as curvature, surface area, volume, and/or the like. In another non-limiting example, one or more cardiac feature datums may include one or more texture features (i.e., characteristics related to the texture or pattern within cardiac tissues, as seen ultrasound image 104), such as gray-level co-occurrence matrix (GLCM) features representing the texture of heart muscle tissue. In another non-limiting example, one or more cardiac feature datums may include one or more orientation features (i.e., characteristics related to the orientation or alignment of cardiac structures), such as the angle or alignment of the septum within the heart. In a further non-limiting example, one or more cardiac feature datums may include one or more edge and boundary features (i.e., Characteristics related to the edges or boundaries between different cardiac structures or tissues), such as edge detection features highlighting the boundary between the myocardium and the cardiac chambers. As an ordinary person skilled in the art, upon reviewing the entirety of this disclosure, will be aware of various cardiac feature datums extracted from ultrasound image 104 in consistent with this disclosure.

With continued reference to FIG. 1, in one or more embodiments, system 100 may include a computer vision model configured to generate 3D organ model 137. A “computer vision model,” for the purpose of this disclosure, is a computation model designed to interpret and make determinations based on visual data. In an embodiment, computer vision model may process ultrasound images, to decide about a scene, space, and/or object in heart. In a non-limiting example, computer vision model may be used for registration of plurality of voxels within a 3D space. In one or more embodiments, registration may include image processing described herein, such as without limitation, object recognition, feature detection, edge/corner detection, and the like. Non-limiting example of feature detection may include scale invariant feature transform (SIFT), Canny edge detection, Shi Tomasi corner detection, and the like. In one or more embodiments, registration may include one or more transformations to orient an ultrasonic image relative to a 3D coordinate system; exemplary transformations include without limitation, homography transforms and affine transforms. In an embodiment, registration of ultrasonic image to a coordinate system may be verified and/or corrected using object identification and/or computer vision, as described above. For instance, and without limitation, an initial registration to two dimensions, represented for instance as registration to the x and y coordinates, may be performed using a two-dimensional projection of points in three dimensions onto the ultrasonic image; however, a third dimension of registration, representing depth and/or a z axis, may be detected by utilizing depth-sensing techniques such as Doppler imaging. Alternatively, the third dimension may be inferred from the known geometry and orientation of the imaging device (e.g., ultrasound sensor 106 of ultrasound imaging device 102), or through the application of one or more machine-learning models trained to interpret depth from the two-dimensional projection.

With continued reference to FIG. 1, processor 110 may use a machine-learning module to implement one or more algorithms or generate one or more machine-learning models, such as a cardiac modeling model to generate 3D organ model 137 of heart. However, the machine-learning module is exemplary and may not be necessary to generate one or more machine-learning models and perform any machine-learning described herein. In one or more embodiments, one or more machine-learning models may be generated using training data. Training data may include inputs and corresponding predetermined outputs so that a machine-learning model may use correlations between the provided exemplary inputs and outputs to develop an algorithm and/or relationship that then allows machine-learning model to determine its own outputs for inputs. Training data may contain correlations that a machine-learning process may use to model relationships between two or more categories of data elements. Exemplary inputs and outputs may come from a database, such as any database described in this disclosure, or be provided by a user. In other embodiments, a machine-learning module may obtain a training set by querying a communicatively connected database that includes past inputs and outputs. Training data may include inputs from various types of databases, resources, and/or user inputs and outputs correlated to each of those inputs so that a machine-learning model may determine an output. Correlations may indicate causative and/or predictive links between data, which may be modeled as relationships, such as mathematical relationships, by machine-learning models, as described in further detail below. In one or more embodiments, training data may be formatted and/or organized by categories of data elements by, for example, associating data elements with one or more descriptors corresponding to categories of data elements. As a non-limiting example, training data may include data entered in standardized forms by persons or processes, such that entry of a given data element in a given field in a form may be mapped to one or more descriptors of categories. Elements in training data may be linked to descriptors of categories by tags, tokens, or other data elements. In a further embodiment, training data may include previous outputs such that one or more machine-learning models iteratively produces outputs.

Still referring to FIG. 1, machine-learning module may be used to generate anatomy modeling model and/or any other machine-learning models, such as, shape identification model as described in further detail below, using training data. Cardiac modeling model may be trained by correlated inputs and outputs of training data. Training data may be data sets that have already been converted from raw data whether manually, by machine, or any other method. In an embodiment, generating 3D organ model 137 of heart may include receiving anatomy training data, wherein the anatomy training data may include a plurality of ultrasound images as input and a plurality of 3D organ models as output. In one or more embodiments, anatomy training data may be received from cardio database 116 or other databases. In other cases, anatomy training data may be collected by a data acquisition unit from external sources such as one or more medical equipment's e.g., imaging devices or diagnostic tools, wherein the data acquisition may be configured as an intermediary between the data source and machine-learning module. In one or more embodiments, anatomy training data may include a plurality of ultrasound images correlated to a plurality of 3D organ models. In one or more embodiments, a particular ultrasound image within anatomy training data may be correlated to a particular 3D organ model 137. In one or more embodiments, anatomy training data may further include a plurality of ultrasound images correlated to a plurality of cardiac models. In an embodiment, a particular ultrasound image may be correlated to a particular cardiac model. In one or more embodiments anatomy training data may include TEE diagrams, Cardiac CTs, ECG signals and/or ultrasonic images as an input and correlated 3D representations of heart.

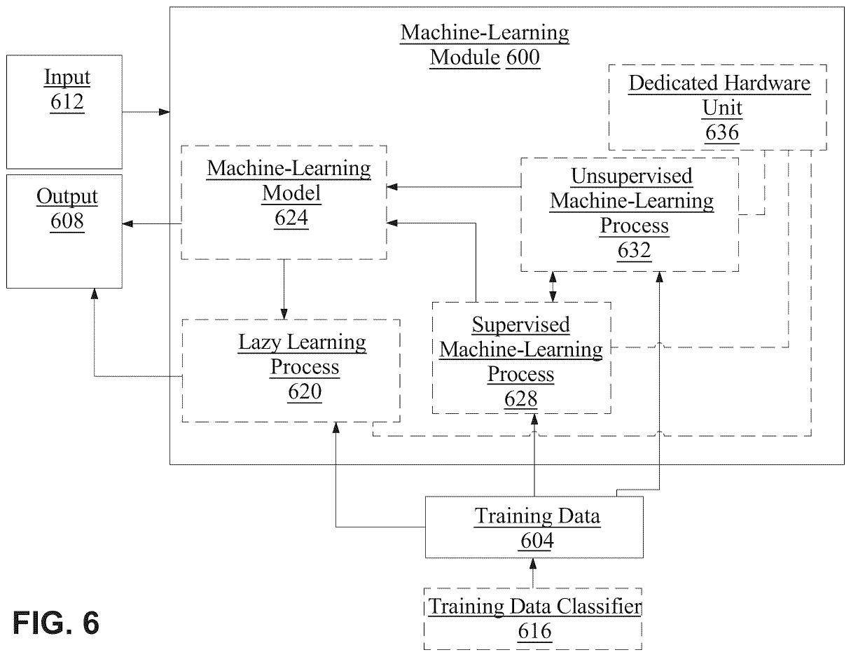

With continued reference to FIG. 1, in an embodiment, anatomy modeling model may include a deep neural network (DNN). As used in this disclosure, a “deep neural network” is defined as a neural network with two or more hidden layers. Neural network is described in further detail below with reference to FIGS. 6-8. In a non-limiting example, anatomy modeling model may include a convolutional neural network (CNN). Generating 3D organ model 137 of heart may include training CNN using anatomy training data and generating 3D organ model 137 as a function of ultrasound images using trained CNN. A “convolutional neural network,” for the purpose of this disclosure, is a neural network in which at least one hidden layer is a convolutional layer that convolves inputs to that layer with a subset of inputs known as a “kernel,” along with one or more additional layers such as pooling layers, fully connected layers, and the like. In one or more embodiments, CNN may include, without limitation, a deep neural network (DNN) extension. Mathematical (or convolution) operations performed in the convolutional layer may include convolution of two or more functions, where the kernel may be applied to input data e.g., ultrasound images through a sliding window approach. In one or more embodiments, convolution operations may enable processor 110 to detect local/global patterns, edges, textures, and any other cardiac feature datums described herein within each ultrasound image. Cardiac feature datums may be passed through one or more activation functions, such as without limitation, Rectified Linear Unit (ReLU), to introduce non-linearities into the processing step of generating 3D organ model 137 of heart. Additionally, or alternatively, CNN may also include one or more pooling layers, wherein each pooling layer is configured to reduce the dimensionality of input data while preserving essential features within the input data. In a non-limiting example, CNN may include one or more pooling layer configured to reduce the spatial dimensions of cardiac feature datum maps by applying downsampling, such as max-pooling or average pooling, to small, non-overlapping regions of one or more cardiac feature datums.

Still referring to FIG. 1, CNN may further include one or more fully connected layers configured to combine cardiac feature datums extracted by the convolutional and pooling layers. In one or more embodiments, one or more fully connected layers may allow for higher-level pattern recognition. In a non-limiting example, one or more fully connected layers may connect every neuron (i.e., node) in its input to every neuron in its output, functioning as a traditional feedforward neural network layer. In one or more embodiments, one or more fully connected layers may be used at the end of CNN to perform high-level reasoning and produce the final output such as, without limitation, a 3D organ model 137 of heart. Further, each fully connected layer may be followed by one or more dropout layers configured to prevent overfitting, and one or more normalization layers to stabilize the learning process described herein.

With continued reference to FIG. 1, CNN may further include a 3D CNN, wherein the 3D CNN, unlike standard 2D CNN, may include utilization of one or more 3D convolutions which allow them to directly process 3D data, thereby enabling processor 110 to generate 3D structures such as 3D organ model 137 of heart using the 3D CNN. In a non-limiting example, 3D CNN may include one or more 3D filters (i.e., kernels) that move through the ultrasound images in three dimensions and capturing spatial relationships in x, y, and z axis. Similar to 3D convolutions, 3D CNN may further include one or more 3D pooling layers that may be used to reduce the dimensionality of ultrasonic images while preserving cardiac feature datums as described above. Additionally, or alternatively, an encoder-decoder structure may be implemented (extended to 3D), by processor 110, in 3D CNN, wherein the encoder-decoder structure includes an encoding path that captures the context and a decoding path that enables precise localization in a same manner as U-net as described above. Such encoder-decoder structures may also include a plurality of skip connections, allowing 3D CNN to use information from multiple resolutions to improve the process of generating 3D organ model 137 of heart.

With continued reference to FIG. 1, in an embodiment, training the cardiac modeling model (i.e., CNN) may include selecting a suitable loss function to guide the training process. In a non-limiting example, a loss function that measures the difference between the predicted 3D VORs and the ground truth 3D structure e.g., CT-based anatomical object models may be used, such as, without limitation, mean squared error (MSE) or a custom loss function may be designed for one or more embodiments described herein. Additionally, or alternatively, optimization algorithms, such as stochastic gradient descent (SGD), may then be used to adjust the anatomy modeling model's parameters to minimize such loss. In a further non-limiting embodiment, instead of directly predicting 3D organ model 137, cardiac modeling model may be trained as a regression model to predict embedded values described herein for each voxel of plurality of voxels within a 3D grid. Additionally, CNN may be extended with additional deep learning techniques, such as recurrent neural networks (RNNs) or attention mechanism, to capture additional features and/or data relationships within data input. These extensions may further enhance the accuracy and robustness of the anatomical object modeling.

With continued reference to FIG. 1, in one or more embodiments, generating at least a 3D organ model 137 may include extracting at least a cardiac feature datum 138 from at least an ultrasound image 104 and segmenting the at least an ultrasound image 104 into a plurality of image segments. As a non-limiting example, extracting cardiac feature datum 138 may include structural elements like left atrium, left atrial appendage, mitral valve, aortic valve, ventricular walls, and the like. In one or more embodiments, extracting a cardiac feature datum 138 may include isolating and identifying cardiac feature datum 138 from ultrasound image 104 using image processing or machine-learning techniques. For the purposes of this disclosure, an “image segment” is a section of an ultrasound image. In one or more embodiments, image segment 142 may include a section of an ultrasound image that has been partitioned based on shared visual or anatomical properties (e.g., cardiac feature datum 138). In a non-limiting example, in a 2D image, segmenting ultrasound image 104 may include all the pixels that make up cardiac feature datum 138, while in a 3D context, it would encompass all the voxels (3D pixels) that constitute cardiac feature datum 138. In one or more embodiments, processor 110 may segment 3D organ model 137 and segmenting 3D organ model 137 may include extracting cardiac feature datum 138 from 3D organ model 137 and segmenting 3D organ model 137 as a function of cardiac feature datum 138.

With continued reference to FIG. 1, processor 110 may be configured to segment ultrasound image 104 to isolate cardiac features and record them as cardiac feature datum 138. In one or more embodiments, only portions of a patient's heart relative to implementation of a cardiovascular device may be contained whereas other portions may be removed. In one or more embodiments,

With continued reference to FIG. 1, in one or more embodiments, processor 110 may be configured to analyze ultrasound image and/or 3D organ model 137 using machine vision system to determine cardiac feature datum 138. For the purposes of this disclosure, a “machine vision system” is a type of technology that enables a computing device to inspect, evaluate and identify still or moving images. For example, in one or more embodiments a machine vision system may be used for world modeling or registration of objects within a space. In one or more embodiments, registration may include image processing, such as without limitation, object recognition, feature detection, edge/corner detection, and the like. Non-limiting example of feature detection may include scale invariant feature transform (SIFT), Canny edge detection, Shi Tomasi corner detection, and the like. In one or more embodiments, a machine vision process may operate image classification and segmentation models, such as without limitation, by way of machine vision resource (e.g., OpenMV or TensorFlow Lite). A machine vision process may detect motion, for example by way of frame differencing algorithms. A machine vision process may detect markers, for example blob detection, object detection (e.g., cardiac feature datum 138), face detection, and the like.

With continued reference to FIG. 1, in one or more embodiments, registration may include one or more transformations to orient a camera frame (or an image or video stream) relative to a three-dimensional coordinate system; exemplary transformations include without limitation, homography transforms and affine transforms. In an embodiment, registration of first frame to a coordinate system may be verified and/or corrected using object identification and/or computer vision, as described above. For instance, and without limitation, an initial registration to two dimensions, represented for instance as registration to the x and y coordinates, may be performed using a two-dimensional projection of points in three dimensions onto a first frame, however. A third dimension of registration, representing depth and/or a z axis, may be detected by comparison of two frames; for instance, where first frame includes a pair of frames captured using a pair of cameras (e.g., stereoscopic camera also referred to in this disclosure as stereo-camera), image recognition and/or edge detection software may be used to detect a pair of stereoscopic views of images of an object; two stereoscopic views may be compared to derive z-axis values of points on object permitting, for instance, derivation of further z-axis points within and/or around the object using interpolation. This may be repeated with multiple objects in field of view, including without limitation, environmental features of interest identified by object classifier and/or indicated by an operator. In an embodiment, x and y axes may be chosen to span a plane common to two cameras used for stereoscopic image capturing and/or an xy plane of a first frame; a result, x and y translational components and ø may be pre-populated in translational and rotational matrices, for affine transformation of coordinates of object, also as described above. Initial x and y coordinates and/or guess at transformational matrices may alternatively or additionally be performed between first frame and second frame, as described above. For each point of a plurality of points on object and/or edge and/or edges of object as described above, x and y coordinates of a first stereoscopic frame may be populated, with an initial estimate of z coordinates based, for instance, on assumptions about object, such as an assumption that ground is substantially parallel to an xy plane as selected above. Z coordinates, and/or x, y, and z coordinates, registered using image capturing and/or object identification processes as described above may then be compared to coordinates predicted using initial guess at transformation matrices; an error function may be computed using by comparing the two sets of points, and new x, y, and/or z coordinates, may be iteratively estimated and compared until the error function drops below a threshold level.

With continued reference to FIG. 1, in one or more embodiments, segmenting an ultrasound image 104 and/or 3D organ model 137 may include training a segmentation model with segmentation training data, wherein the segmentation training data may include exemplary ultrasound image and/or 3D organ model 137 correlated to exemplary segmented ultrasound image and/or 3D organ model 137 and segmenting a ultrasound image 104 and/or 3D organ model 137 using the trained segmentation model. For the purposes of this disclosure, a “segmentation model” is a machine-learning or deep learning model designed to partition an image into multiple segments or regions, each corresponding to different objects or parts of an object within the image. In one or more embodiments, segmentation model may assign a label to each pixel in an image (e.g., ultrasound image 104 and/or 3D organ model 137) such that pixels with the same label share certain characteristics, such as belonging to the same cardiac feature datum 138 or region. As a non-limiting example, segmentation model may include a neural network. For the purposes of this disclosure, “segmentation training data” is data containing correlations that a machine-learning process may use to model relationships between echo depth maps and segmented echo depth maps. In a non-limiting example, a segmentation model may analyze ultrasound image 104 and/or 3D organ model 137 to identify and delineate the boundaries of cardiac feature datum 138. This may include finding the set of coordinates {(Xᵢ, Yᵢ)} that represent the pixels or voxels making up the cardiac feature datum 138. In one or more embodiments, processor 110 may segment ultrasound image and/or 3D organ model 137 based on cardiac feature datum 138. In one or more embodiments, segmentation training data may be stored in cardio database 116. In one or more embodiments, segmentation training data may be received from one or more users, cardio database 116, external computing devices, and/or previous iterations of processing. As a non-limiting example, segmentation training data may include instructions from a user, who may be an expert user, a past user in embodiments disclosed herein, or the like, which may be stored in memory and/or stored in cardio database 116, where the instructions may include labeling of training examples. In one or more embodiments, segmentation training data may be updated iteratively on a feedback loop. As a non-limiting example, processor 110 may update segmentation training data iteratively through a feedback loop as a function of output of feature extraction model, ultrasound image 104, 3D organ model 137, and the like. In one or more embodiments, processor 110 may be configured to generate segmentation model. In a non-limiting example, generating segmentation model may include training, retraining, or fine-tuning segmentation model using segmentation training data or updated segmentation training data. In one or more embodiments, processor 110 may be configured to segment ultrasound image 104 and/or 3D organ model 137 using segmentation model (i.e., trained or updated segmentation model).

With continued reference to FIG. 1, in one or more embodiments, generating at least a 3D organ model 137 may include generating a 3D point cloud 140 as a function of a plurality of image segments and generating a 3D mesh model 144 of at least a 3D organ model 137 as a function of the 3D point cloud 140. For the purposes of this disclosure, a “three-dimensional point cloud” is a collection of data points in space, each represented by its x, y, and z coordinates. In one or more embodiments, 3D point cloud 140 may capture the geometry of cardiac feature datums, providing a comprehensive 3D representation. In one or more embodiments, the construction of 3D point cloud 140 may integrate cardiac feature datums and/or image segments from multiple frames. In a non-limiting example, when cardiac feature datums and/or image segments (z) is added to pixel coordinates to convert them into 3D points (x, y, z), all the 3D points can be aggregated to form 3D point cloud 140. In one or more embodiments, 3D organ model 137 may include 3D mesh model 144. For the purposes of this disclosure, a “three-dimensional mesh model” is a mathematical and geometric representation of the surface of a three-dimensional object. In one or more embodiments, 3D mesh model 144 may be constructed using a network of vertices, edges, and faces. In one or more embodiments, vertices may define points in 3D space, the edges may connect pairs of vertices, and the faces, in the form of triangles or quadrilaterals, may create a polygonal surface that represents the shape of the object (heart). In one or more embodiments, processor 110 may generate a mesh representing cardiac shape as a function of 3D voxel occupancy representation. Processor 110 may be configured to display, using display, a mesh to a user. Additional disclosure related to generation of 3D organ model 137 may be found in U.S. Nonprovisional application Ser. No. 18/787,196, filed on Jul. 29, 2024, and entitled “APPARATUS AND METHOD FOR OBJECT POSE ESTIMATION IN A MEDICAL IMAGE,” having an attorney docket number of 1518-163USU1, and U.S. Nonprovisional application Ser. No. 18/938,980, filed on Nov. 6, 2024, and entitled “APPARATUS AND METHOD OF DETERMINING A CARDIAC IMPLANT SIZE,” having an attorney docket number of 1518-171U SU 1, the entirety of which is incorporated herein by reference.

With continued reference to FIG. 1, in one or more embodiments, 3D organ model 137 may include a statistical shape model (SSM 146). As used in this disclosure, a “statistical shape model (SSM)” is a data structure representing, including, and/or utilizing a mathematical model that captures principal modes of variation in shape across a population of cardiac anatomies. SSM 146 captures a plurality of heart models associated with a plurality of patients. In one or more embodiments, SSM 146 may be used to capture the variability in anatomical structures among different patients; for instance, SSM 146 of the human heart may be constructed from a plurality of heart images of a plurality of individuals. In one or more embodiments, 3D organ model 137 generated by SSM model may capture the “average” heart shape and main ways in which heart shapes may vary among the plurality of individuals. In one or more embodiments, 3D organ model 137 generated by SSM model may capture the “average” of the plurality of anatomical objects in which anatomical objects may vary among plurality of individuals. In one or more embodiments, SSM 146 may be generated by processor 110 as a function of a set of labeled example shapes, each in a form of point-based representations (3D point clouds) or meshes. In one or more embodiments, example shapes may be represented in a 3D voxel occupancy representation (VOR).

With continued reference to FIG. 1, generating 3D organ model 137 may include generating 3D organ model 137 using a point completion model. For the purposes of this disclosure, a “point completion model” is an algorithm that is configured to fill in missing data within a point cloud. In some embodiments, point completion model may include one or more deep learning models. In some embodiments, point completion model may include point-based methods. Point based methods may include modeling each point individually using multilayer perception (MLP) layers. In some embodiments, features may be learned from raw input point cloud data. This may reduce reliance on prior information and/or manually set parameters. In some embodiments, point-based methods may use an encoder-decoder architecture. In some embodiments, encoder-decoder may include an end-to-end cascaded neutral network. In some embodiments, point completion model may use a selective focus approach. A selective focus approach may include an attention mechanism. Attention is an adaptive mechanism that is used to capture information and assign higher weights to important data. In some embodiments, point completion model may include an unsupervised 3D point cloud capsule network that uses autoencoders to process sparse point clouds and preserve the spatial arrangement. In some embodiments, attention mechanisms may be used to enhance the resolution and fill in missing parts. In some embodiments, point completion model may use a view-guided approach. Relying on a single view may be susceptible to scene and temporal limitations, which can result in lost details. In some embodiments, a view-guided framework (ViPC) that retrieves absent global shape information from alternative single-view images may be used. By including additional single-view images, ViPC may provide global structural prior information for point cloud completion. In some embodiments, point completion model may use a point completion network (PCN)-assisted approach. Some deep-learning methods usually discretize 3D data into voxels that act directly on the convolution operations. Instead, in some embodiments, a multi-stage point cloud completion network (MSPCN) with crucial set oversight, incorporating a cascading upsampling module to achieve high-resolution outcomes gradually may be used. The vital sets for each stage may be used for oversight, thereby generating more informative and valuable intermediary outputs for the subsequent stage. In some embodiments, a skeleton-bridged PCN (SK-PCN) to complement shapes by locally scanning them may be used. Initially the SK-PCN may forecast their 3D skeleton to attain a universal structure, and complete surfaces by learning the shifts in the skeleton points. In some embodiments a point enhancement network (M E-PCN) may use the “void” in the 3D shape space to approximate rough but complete and coherent surface points and then produce fine-grained surface details in the refinement stage. A local-to-local strategy may be used in some embodiments, and an attentional point cloud aggregation module may be used to aggregate local scans to complete point clouds.

With continued reference to FIG. 1, additionally, 3D reconstruction of ultrasound images into 3D organ models is described in a number of co-owned patent applications listed below. Each of these applications are incorporated herein by reference in their entirety: U.S. Nonprovisional application Ser. No. 18/818,034, filed on Aug. 28, 2024, and entitled “APPARATUS AND METHODS FOR GENERATING A THREE-DIMENSIONAL (3D) MODEL OF AN ANATOMICAL OBJECT VIA MACHINE-LEARNING,” having an Attorney Docket No. 1518-103USC2, which is a continuation-in-part of Non-provisional application Ser. No. 18/750,411 filed on Jun. 21, 2024, and entitled “APPARATUS AND METHODS FOR GENERATING A THREE-DIMENSIONAL (3D) MODEL OF AN ANATOMICAL OBJECT VIA MACHINE-LEARNING,” having an Attorney Docket No. 1518-103USC1, which is a continuation of Non-provisional application Ser. No. 18/376,688 filed on Oct. 4, 2023, and entitled “APPARATUS AND METHODS FOR GENERATING A THREE-DIMENSIONAL (3D) MODEL OF CARDIAC ANATOMY VIA MACHINE-LEARNING,” having an Attorney Docket No. 1518-103USU1, the entirety of which are incorporated herein by reference and U.S. Nonprovisional application Ser. No. 18/817,870, filed on Aug. 28, 2024, and entitled “APPARATUS AND METHODS FOR SYNTHESIZING MEDICAL IMAGES,” having Attorney Docket No. 1518-104USC1, which is a continuation-in-part of Non-provisional application Ser. No. 18/509,520, filed on Nov. 15, 2023, and entitled “APPARATUS AND METHODS FOR SYNTHESIZING MEDICAL IMAGES,” having an Attorney Docket No. 1518-104USU1, the entirety of which are incorporated herein by reference and U.S. Non-provisional application Ser. No. 18/818,152, filed on Aug. 28, 2024, and entitled “APPARATUS AND METHOD FOR GENERATING A THREE-DIMENSIONAL (3D) MODEL OF CARDIAC ANATOMY BASED ON MODEL UNCERTAINTY,” having Attorney Docket No. 1518-105USC1, which is a continuation-in-part of Non-provisional application Ser. No. 18/426,604, filed on Jan. 30, 2024, and entitled “APPARATUS AND METHOD FOR GENERATING A THREE-DIMENSIONAL (3D) MODEL OF CARDIAC ANATOMY BASED ON MODEL UNCERTAINTY,” having Attorney Docket No. 1518-105USU1, the entirety of which are incorporated herein by reference and U.S. Non-provisional application Ser. No. 18/818,311 filed on Aug. 28, 2024, and entitled “APPARATUS AND METHOD FOR GENERATING A THREE-DIMENSIONAL (3D) MODEL OF CARDIAC ANATOMY WITH AN OVERLAY,” having Attorney Docket No. 1518-110USC1, which is a continuation-in-part of Non-provisional application Ser. No. 18/395,087 filed on Dec. 22, 2023, and entitled “APPARATUS AND METHOD FOR GENERATING A THREE-DIMENSIONAL (3D) MODEL OF CARDIAC ANATOMY WITH AN OVERLAY,” having Attorney Docket No. 1518-110USU1, the entirety of which are incorporated herein by reference and U.S. Non-provisional application Ser. No. 18/648,176 filed on Apr. 26, 2024, and entitled “APPARATUS AND METHODS FOR VISUALIZATION WITHIN A THREE-DIMENSIONAL MODEL USING NEURAL NETWORKS,” having Attorney Docket No. 1518-116USU1, the entirety of which is incorporated herein by reference.

With continued reference to FIG. 1, in one or more embodiments, generating 3D organ model 137 representative of patient's heart may include receiving a generic 3D model 148. A “generic 3D model” for the purposes of this disclosure is a three-dimensional representation of a heart not associated to any individual. For example, and without limitation, generic 3D model 148 may include a digitally created three-dimensional model of a heart. In one or more embodiments, generic 3D model 148 may include density, measurements and/or the like of determined averages of individuals' hearts. In one or more embodiments, generic 3D model 148 may include an aggregation of a plurality of ultrasound images that are used to create a 3D model representative of the aggregate. In one or more embodiments, generic 3D model 148 may be received from any database as described in this disclosure. In one or more embodiments, generic 3D model 148 may include a 3D organ model 137 representing values of an average organ based on a plurality of ultrasound images. In one or more embodiments, generic 3D model 148 may include a digital representation of an organ having no issues. For example, and without limitation, generic 3D model 148 may include a digital representation of a heart having no blockages within the arteries. In one or more embodiments, generic 3D model 148 may include a digital representation of an organ that is deemed to be a healthy organ. In one or more embodiments, generic 3D model 148 may be used as a reference for other digital models representing a heart. In one or more embodiments, generic 3D model 148 may include one or more values of an organ that is deemed to be healthy. In one or more embodiments, the values may include tissue density, valve diameter, heart shape and/or any other values that are described in reference to 3D organ model 137. In one or more embodiments, values may be categorized based on cardiac features, wherein each cardiac feature within generic 3D model 148 may contain its own values. In one or more embodiments, generic 3D model 148 may contain generic cardiac feature datum, wherein generic cardiac feature datum includes cardiac feature datum of generic 3D model 148.

With continued reference to FIG. 1, processor 110 may be configured to generate 3D organ model 137 as a function of generic model. In one or more embodiments, processor may be configured to receive generic 3D model 148 and refine generic 3D model 148 to create 3D organ model 137.

With continued reference to FIG. 1, processor 110 may be configured to generate a refined organ model as a function of refinement. As used in this disclosure, a “refined organ model” refers to an organ model customized to reflect a patient's organ. For example, and without limitation, refined organ model may include 3D organ model 137. In one or more embodiments, generic 3D model 148 may be refined to create 3D organ model 137 and/or refined model. In an embodiment, refined organ model may be derived from generic 3D model 148 and adjusted based on sensor data and/or ultrasound images. In such embodiment, refined organ model may include a deformed organ model as described above. In a non-limiting example, 3D VOR may indicate a need of adjustment to generic 3D model 148 to match a subject's unique geometry. SSM may then be configured to generate refined organ model that accurately capture such specific organ anatomy based on generic 3D model 148 and 3D VOR. In other cases, generic 3D model 148 may not need any refinement; for instance, and without limitation, if generic 3D model 148 already align perfectly with subject's organ, no deformation or adjustment would be necessary, thereby resulting in refined organ model and/or 3D organ model 137 that is identical to generic 3D model 148.

Still referring to FIG. 1, in some cases, the refinement process may also include the incorporation of more detailed features and textures based on sensor data such as ultrasound images thereof, enhancing the realism and specificity of generic 3D model 148. In an embodiment, SSM may be integrated with one or more additional models such as, without limitation, texture models, appearance models, or functional models to generate refined organ model and/or 3D organ model 137. In some cases, such integration may result in refined organ model that reflect not just the geometry but also the biomechanical properties or blood flow dynamics within organ. In a non-limiting example, texture of the myocardium may be modeled, by integrating texture models with SSM, to represent the fibrous nature of the heart muscle. In another non-limiting example, appearance of blood vessels, including color variations and translucency, may be modeled, by integrating appearance models with SSM.

With continued reference to FIG. 1, processor 110 may be configured to generate a set of shape parameters based on set of ultrasound images. As used in this disclosure, a “set of shape parameters” refers to a collection of numerical values or descriptors that quantitatively represent the geometric or morphological characteristics of a structure. In some embodiments, a set of shape parameters may represent the shape of a structure. In a non-limiting example, set of shape parameters may include information and/or metadata calculated, determined, and/or extracted from set of ultrasonic images, such as, dimensions, angles, curvatures, surface areas, texture, symmetry, and/or the like. In other embodiments, processor may be configured to parameterize features (e.g., edges, textures, contours, and any other characteristics that describe the shape structure) extracted from set of ultrasound images using CNN described herein. Such parameterization may involve processor to derive one or more shape parameters including one or more morphological descriptors that quantitatively describe structure based on extracted features. In some cases, processor may be configured to use principal component analysis (PCA) to reduce the dimensionality of set of shape parameters, allowing processor to focus on the most informative shape parameters of set of shape parameters in further processing steps described below.

With continued reference to FIG. 1, in a non-limiting example, set of shape parameters may be generated based on set of ultrasound images using machine learning model such as, without limitation, a shape identification model. Generating set of shape parameters may include receiving structure training data, wherein the structure training data may include a plurality of image sets as inputs correlated to a plurality of shape parameter sets as outputs. In some cases, structure training data may be received from a data store. For example, and without limitation, structure training data may be used to show each ultrasonic image may indicate a particular set of shape parameters. In some embodiments, structure training data may include historical ultrasonic images correlated with historical computed tomography scan data. Such a training dataset may be used to train shape identification model to generate a set of shape parameters representing a structure's shape as a function of a set of ultrasonic images, which may be input into the model to receive, as an output, a set of shape parameters. Shape identification model may be trained by processor, using structure training data. Additionally, structure training data may include previously input image sets and their corresponding shape parameter outputs. Shape identification model may be iterative such that outputs may be used as future inputs of shape identification model. This may allow the shape identification model to evolve. Processor may be further configured to generate set of shape parameters as a function of set of images using the trained shape identification model.

Still referring to FIG. 1, generating set of shape parameters may include performing image processing/segmentation techniques, as described above, prior to implementation of shape identification model to optimize performance and runtime of processor and training of model. For example, image segmentation may include normalization and standardization methods performed by computer vision model to ensure that pixel values in images are normalized or standardized to a consistent scale thus aiding convergence during training of shape identification model. Image segmentation may include data augmentation techniques such as rotation, scaling, flipping, and translation to artificially increase the size of the training dataset and improve model generalization. Image segmentation may include image enhancement preprocessing techniques like histogram equalization or contrast stretching to enhance relevant features in the images. Image segmentation may include texture and shape descriptors to extract features beyond pixel values, such as texture and shape descriptors, to capture additional information about structures. Image segmentation may include architecture selection methods, as in experiments with different architectures, such as U-Net, DeepLab, or custom architectures, depending on the complexity and characteristics of the images. Image segmentation may include grid Search or random Search processing methods to systematically explore hyperparameter combinations to find the optimal configuration for a D model. As previously disclosed, image segmentation may include separating specific structures or regions of interest (ROI) from the background or other structures in a given ultrasonic image, wherein a collection of ROIs may also be incorporated by the shape parameter training data/structure training data.

With continued reference to FIG. 1, alternatively, refining generic 3D model 148 may include adjusting generic 3D model 148 based on set of shape parameters. In an embodiment, processor 110 may be configured to map set of shape parameters to SSM. The mapping process may define how generic 3D model 148 should be adjusted to represent specific subject's organ anatomy. In a non-limiting example, shape parameters may include one or more numeric values indicating a particular thick ventricular wall, processor 110 may configure SSM to adjust generic 3D model 148 to reflect such characteristic. In an embodiment, generating refined organ model may involve generating a 3D mesh or grid that accurately represents the shape defined by set of shape parameters; for instance, and without limitation, processor 110 may be configured to generate a 3D mesh for left ventricle with vertices and edges positioned according to specific curvature and thickness defined by set of shape parameters using SSM.

With continued reference to FIG. 1, processor 110 may be configured to input generic 3D model 148 into one or more machine-learning models, such as for example, cardiac modeling model, as described in this disclosure to create 3D organ model 137. In one or more embodiments, processor 110 may utilize a machine vision system to modify generic 3D model 148 to create 3D organ model 137. In one or more embodiments, processor 110 may be configured to identify one or more anomalies within ultrasound image. An “anomaly” for the purposes of this disclosure refers to a datapoint of set of data points that deviates from an expected pattern or result. For example, and without limitation, anomaly within ultrasound image 104 may include the presence of a lump that may not ordinarily be present within a healthy heart. In another non-limiting example, anomaly may include a particular size of the heart, or various portions thereof that may not conform to sizes typically associated with a healthy heart. In one or more embodiments, anomaly may include any change in density, size, thickness and/or the like of various feature datum within ultrasound image in comparison to generic 3D model 148. In one or more embodiments, anomalies may be used to compare ultrasound image 104 to generic 3D model 148 and determine differences in distances, densities and/or the like between a patient's heart and that of a Generic model. In one or more embodiments, generic 3D model 148 may include generic cardiac feature Datum, wherein generic cardiac feature datum includes cardiac feature datum of generic 3D model 148. In one or more embodiments, processor 110 may identify anomalies within ultrasound image by comparing cardiac feature datum to generic cardiac feature datum. In an embodiment, differences in values between a particular cardiac feature datum and generic cardiac feature datum may indicate the presence of one or more anomalies. In one or more embodiments, processor 110 may be configured to modify generic 3D model 148 based on the identified anomalies to generate 3D organ model 137. In one or more embodiments, generic 3D model 148 may be modified in any way as described in this disclosure. In one or more embodiments, generic 3D model 148 may be modified in any way similar to the generation of 3D organ model 137 as described in this disclosure. In one or more embodiments, processor may be configured to generate 3D organ model 137 by adjusting values within generic 3D model 148, such as for example, a thickness, density, contours, surfaces and/or other geometric representations. In one or more embodiments, creating 3D organ model 137 may include modifying one or more voxels and/or 3D grid embedded values within generic 3D model 148. In one or more embodiments, generic 3D model 148 may include a plurality fo cardiac features wherein only cardiac features identified as anomalies may be modified. In an embodiment, features not identified within ultrasound images may be given generic cardiac feature datums. In an embodiment, modification of generic 3D model 148 may ensure that values not identified and/or cardiac feature datums not identified may instead be replaced with generic features from within generic 3D model 148.

With continued reference to FIG. 1, an anomaly may include a spatial distortion. A “Spatial distortion” for the purposes of this disclosure refers to a change in measurement between the patient's heart and a generic heart with generic 3D model 148. For example, and without limitation, spatial distortion may include a change in length or radius of a ventricle calculated from a comparison between cardiac feature datum and generic cardiac feature datum. In one or more embodiments, spatial distortion may indicate that a sizing of the patient's heart and/or portions thereof may be different from that of Generic 3D model. In one or more embodiment, spatial distortion may be used to generate 3D organ model 137, wherein a 3D grid for example may be modified. In one or more embodiments, spatial distortions may indicate changes in measurement between ultrasound image and generic 3D model 148, wherein generic 3D model 148 may require modification to match the anatomy of the patient. In one or more embodiments, anomaly may include a spatial distortion of at least one cardiac feature of the patient. This may include for example, a difference in measurement of a ventricle, a difference in measurement of a heart chamber and/or the like. In one or more embodiments, 3D organ model 137 may be generated by identifying anomalies within ultrasound image 104 and modifying generic 3D model 148 to include and/or account for the anomalies.

With continued reference to FIG. 1, receiving 3D organ model 137 may include generating 3D organ model 137 as a function of one or more ultrasound images received from TEE system, intracardiac echocardiography system and/or any ultrasound imaging device as described in this disclosure.

With continued reference to FIG. 1, processor 110 is configured to generate treatment guidance 150. For the purposes of this disclosure, “treatment guidance” is information that provides procedural direction to a user based on a treatment plan. Generating treatment guidance 150 includes locating a puncture site 152 on 3D organ model 137. A “puncture site” for the purposes of this disclosure refers to a portion of 3D organ model that is suitable for perforation. In one or more embodiments, puncture site 152 may include a portion of 3D organ model 137 that is suitable for a medically related procedure related to puncturing an organ. For example, and without limitation, puncture site 152 may include a location suitable for transseptal puncture. “Transseptal puncture” for the purposes of this disclosure is medical procedure in which a hole is created in the interatrial septum to transition from a right atrium to a left atrium of an individual's heart. In one or more embodiments, puncture site 152 may include a location suitable for transseptal puncture. In one or more embodiments, numerous cardiac interventions, including left atrial appendage closure, mitral valve repair, left-sided ablations, and left-sided lead placements, require access to the left side of the heart. In one or more embodiments, puncture site 152 may include a location in which a hole and/or perforation may be made on a subject to access the left side of the heart. In one or more embodiments, puncture site 152 may represent a location on a 3D organ model 137 that is representative of a subject's organ suitable for perforation. In one or more embodiments, puncture site 152 may be used to assist in medical procedures by providing a particular location of 3D organ model 137 suitable for perforation, wherein the location is representative of a location on a subject's heart suitable for perforation. In one or more embodiments, an accurately selected puncture site 152 can lead to procedural inefficiencies, increased procedure times, and higher risks for complications. This is particularly evident in procedures like left atrial appendage closure, where an inappropriate puncture site 152 can extend the procedure from 30 minutes to several hours. In one or more embodiments, puncture site 152 may include a location in which a catheter and/or ultrasound sensor may be inserted to gain access to the left side of the heart. In one or more embodiments, puncture site 152 may include three dimensional coordinates, such as for example, coordinates on an X-Y-Z reference system. In one or more embodiments, puncture site 152 may include a location relative to 3D organ model 137. In one or more embodiments, puncture site 152 includes a location coordinate on 3D organ model 137. A “location coordinate” for the purposes of this disclosure refers to a set of alphanumeric numbers or characters that define a specific point or location within a given coordinate system. For example, and without limitation, location coordinate may include X, Y and Z coordinates. In one or more embodiments, location coordinates may include coordinates within a polar coordinate system, a spherical and/or cylindrical coordinate system and/or the like. In one or more embodiments, puncture site 152 may include location coordinates on and/or relative to 3D organ model 137, wherein location coordinates may specific a particular point and/or area on 3D organ model 137. In one or more embodiments, location coordinates may include a particular distance from a given reference frame, such as for example, a bottom right corner of 3D organ model 137. In one or more embodiments, 3D organ model 137 may be mapped onto a coordinate system, wherein location coordinate may specify location of puncture site 152 relative to the coordinate system.

With continued reference to FIG. 1, processor 110 is configured to generate treatment guidance 150 as a function of treatment plan 164. Puncture site 152 may be located as a function of a treatment plan 164. A “treatment plan” for the purposes of this disclosure refers to a procedure or strategy involving cardiovascular intervention. For example, and without limitation, treatment plan 164 may include cardiac interventions, such as but not limited to, including left atrial appendage closure, mitral valve repair, left-sided ablations, left-sided lead placement and/or the like. In one or more embodiments, treatment plan 164 may include any medical procedure and/or treatment involving the heart. In one or more embodiments, treatment plan 164 may include a particular portion of the heart that a medical professional believes requires treatment, such as but not limited to, left atrial appendage, mitral valve, pulmonary veins and/or the like. In one or more embodiments, treatment plan 164 includes an area of interest 168. An “area of interest” for the purposes of this disclosure refers to a portion of the heart that requires attention. For example, and without limitation, area of interest 168 may include a left atrium, a left atrial appendage, pulmonary veins, mitral valves, a left ventricle, an interatrial septum and/or the like. In one or more embodiments, area of interest 168 may designate a particular area of an area associated with organ model that requires treatment and/or evaluation. In one or more embodiments, area of interest 168 may include location coordinates relative to 3D organ model 137, a particular portion of 3D organ model 137 and/or the like. In one or more embodiments, area of interest 168 may be designated by an individual using a remote device, such as any remote device as described in this disclosure. In one or more embodiments, machine vision system may classify one or more portions of 3D organ model 137, wherein area of interest 168 may include a selection of one or more classified portions of 3D organ model 137. In one or more embodiments, machine vision system may be configured to classify portions of 3D organ model 137, such as but not limited to, left ventricles, right ventricles and/or any other portions of the heart. In one or more embodiments, generic 3D model 148 may include portions already containing labels, wherein area of interest 168 may include a selection of one or more portions of generic 3D model 148 and/or refined 3D model.

With continued reference to FIG. 1, puncture site 152 may be determined by identifying a trans-septum port with 3D organ model 137. In one or more embodiments, trans-septum port refers to a medical access point created through the atrial septum, the wall that separates the left and right atria of the heart. It is most often associated with procedures that require access to the left atrium via the right atrium and/or the like. In one or more embodiments, generic 3D model 148 may include a label and/or location for trans-septum port, wherein refined 3D model may include the refined location of trans-septum port. In one or more embodiments, puncture site 152 may be determined by identifying trans-septum port on 3D organ model 137. In one or more embodiments, processor 110 may use machine vision system to classify and/or identify trans-septum port. In one or more embodiments, puncture site 152 may include a location of trans-septum port on 3D organ model 137.

With continued reference to FIG. 1, treatment plan includes a procedure type. A “procedure type” for the purposes of this disclosure refers to the selection of a medical procedure. In one or more embodiments, procedure type may include the medical procedure that a medical professional is seeking to perform following or during insertion of a catheter through puncture site. In one or more embodiments, procedure type may include but is not limited to, Atrial fibrillation ablation (e.g., pulmonary vein isolation), Left atrial appendage closure (e.g., Watchman device implantation), Mitral valve repair or replacement (e.g., MitraClip placement), Balloon mitral valvuloplasty, Left atrial pressure measurement, Transseptal catheterization for access to the left atrium, Left atrial mapping, Left-sided electrophysiological studies, Left ventricular endocardial pacing lead placement, Transseptal delivery of investigational cardiac devices, Paravalvular leak closure (mitral or aortic), Alcohol septal ablation via left atrial access, Transcatheter mitral valve-in-valve or valve-in-ring procedures, Left atrial thrombus aspiration (rare and selective cases), Cardiac resynchronization therapy with left-sided access and/or the like. In one or more embodiments, procedural type may include any cardiovascular related procedure that is related to or requires transseptal puncture. In one or more embodiments, procedure type may include selection of a particular cardiovascular device that needs to be implanted within a subject.

With continued reference to FIG. 1, a “cardiovascular device” as described in this disclosure refers to a medical device that is used in the management of diseases or conditions affecting the heart. For example, and without limitation, cardiovascular device may include heart valves. In one or more embodiments, cardiovascular device may be used for heart valve repair as described in this disclosure. In one or more embodiments, cardiovascular device may be used to stabilize and reshape the ring around heart valves to restore normal valve function. In one or more embodiments, cardiovascular device may be used to reinforce the areas of a heart valve that may be weakened or damaged. In one or more embodiments, cardiovascular device may be implanted within the heart of a patient to improve the functioning of one or more arteries and/or valves. In one or more embodiments, cardiovascular device may include a stent. A “Stent” as described in this disclosure is a tube configured to be implanted within the heart of a patient and used to keep the arteries of veins open. In one or more embodiments, stent may include a bare-metal stent, a drug-eluding stent, a biodegradable stent, a covered stent, a graft stent, a self-expanding stent, a ballon-expandable sent and/or the like. In one or more embodiments, cardiovascular device may include a heart valve. A “heart valve” as described int this disclosure is prosthetic calve that is configured to replace or repair a damaged heart valve. In one or more embodiments, heart valve may include heart valves, such as but not limited to, tilting disc valves, stented tissue valves, stent less tissue valves, homograft, autografts, transcatheter heart valves, aortic valves, mitral valves, tricuspid valves, pulmonary valves and/or the like.

With continued reference to FIG. 1, procedural type may include device instructions for use (IFU) data. For the purposes of this disclosure, “device Instructions for Use (IFU) data” is data related to procedural guidelines or specifications associated with cardiovascular device. As a non-limiting example, device IFU data may include usage instructions, imaging requirements, and the like. For example, and without limitation, device IFU data may include specific angles for the placement of cardiovascular device on a coronary artery. In one or more embodiments, device IFU data may include instructions on the angle in which cardiovascular device may need to be inserted, instructions on what type of catheter may be required to navigate cardiovascular device and/or the like. For example, a catheter and corresponding cardiovascular device should enter the Left atrial appendage coaxially, meaning aligned with the central axis of the appendage.

With continued reference to FIG. 1, treatment plan 164 may include a catheter selection. A “catheter selection” for the purposes of this disclosure refers to the selection of a particular catheter for us in a procedure associated with the heart. For example, and without limitation, catheter selection may include, but is not limited to, a transseptal needle, a radiofrequency, transseptal needle, a transseptal sheath, a steerable sheath, a circular mapping catheter, an irrigated RF ablation catheter, a cryo-balloon catheter, a left atrial appendage occlusion device, a mitral valve repair catheter, a left ventricular access catheter and/or the like. In one or more embodiments, catheter selection may indicate the area in which puncture site 152 should be located such that perforation does not lead to any medically related issues. In one or more embodiments, catheter selection may be indicative of a particular medical procedure wherein each medical procedure may require a differing location of puncture site 152. For example, and without limitation, access to a left atrium may require a differing puncture site 152 in comparison to a mitral valve.

With continued reference to FIG. 1, in one or more embodiments, catheter selection may include a pliability metric. A “pliability metric” for the purposes of this disclosure refers to a metric quantifying the ability of a catheter to deform. For example, and without limitation, a steel or aluminum catheter may not be pliable, whereas a silicone catheter may allow for pliability. In one or more embodiments, each catheter within catheter selection may include a pliability metric indicating how pliable a particular catheter is. In one or more embodiments, pliability metric may include a numerical range, such as for example, ranging from 1-10, wherein a ‘1’ may indicate a lack of pliability and a 10 may indicate that the catheter can deform with little to no forces applied. In one or more embodiments, pliability metric may include a torque response for each catheter, a flexural modulus (bending stiffness), force-displacement behavior, bend radius and/or the like. In one or more embodiments, pliability metric may be quantified in newtons, pascals, newton-meters, millimeters and/or the like. In one or more embodiments, pliability metric may indicate whether a catheter is capable of deforming on order to access area of interest 168 and/or whether a catheter is not capable of deforming and thus cause perforations within the heart when trying to access area of interest 168. In one or more embodiments, pliability metric may indicate an ability of catheter to navigate through the heart by deforming and navigating around portions of the heart. In one or more embodiments, pliability metric may indicate whether the pliability of catheter allows for manipulation of catheter to prevent perforations while navigating the heart. In one or more embodiments, a low pliability metric may indicate that a catheter is capable of deformation and thus the catheter is capable of navigating through valves, and/or portions fo the heart.

With continued reference to FIG. 1, in some embodiments, catheter selection may include a steerability metric. A “steerability metric,” for the purposes of this disclosure, is a metric quantifying the capability of a catheter to be steered. In some embodiments, a catheter may include a steerable sheath. A steerable sheath may allow for control over catheter manipulation. In some embodiments, catheter may include a steerable catheter. In some embodiments, catheter may include a steerable guidewire. In some embodiments, steerability metric may include one or more degrees of freedom. In some embodiments, degrees of freedom may include a push/pull of the catheter, a rotation of the catheter, a rotation of the catheter tip, and the like.

With continued reference to FIG. 1, processor 110 may locate puncture site 152 as a function of treatment plan 164. In one or more embodiments, each area of interest 168 may be correlated with a specific puncture site, wherein the presence of a particular area of interest 168 may indicate to processor 110 to selection a particular location of puncture site 152. In one or more embodiments, each area of interest 168 may be associated and/or contain a correlated puncture site 152. In one or more embodiments, each area of interest 168 may contain coordinates relative to a generic 3D model 148, wherein puncture site 152 may include coordinates that have been refined and/or adjusted for 3D organ model 137.

With continued reference to FIG. 1, treatment plan 164 may be received by subject, from a medical professional interacting with system 100, extracted from a patient health record, received by remote device such as any remote device as described in this disclosure, received from a database, such as any database as described in this disclosure and/or the like.

With continued reference to FIG. 1, processor 110 may use a machine-learning model such as any machine-learning model as described in this disclosure to locate puncture site 152. In one or more embodiments, a machine-learning model such as puncture machine-learning model 156 may be configured to receive as an input treatment plan 164 and output puncture site 152. In one or more embodiments, puncture site 152 may include location coordinates relative to a generic 3D model 148. In one or more embodiments, processor 110 may then be configured to adjust location coordinates based on changes between generic 3D model 148 and 3D organ model 137. In one or more embodiments, training a machine-learning model to output puncture sites based on one size and shape or a particular organ may increase efficiency and accuracy due to the use of less training data. In one or more embodiments, a machine-learning model such as a puncture machine-learning model 156 may contain parameter values. In one or more embodiments, parameter values may be adjusted during training or pretraining to minimize a loss function. In one or more embodiments, during training, predicted outputs of the machine-learning model are compared to actual outputs wherein the discrepancy between predicted output and actual outputs are measured to minimize a loss function. A loss function also known an “error function” may measure the difference between predicted outputs and actual outputs to improve the performance of the machine-learning model. A loss function may quantify the error margin between a predicted output and an actual output wherein the error margin may be sought to be minimized during the training process. The loss function may allow for minimization of discrepancies between predicted outputs and actual outputs of the machine-learning model. In one or more embodiments, the loss function may adjust parameter values of the machine-learning model. In one or more embodiments, in a linear regression model, parameter values may include coefficient assigned to each feature and the bias term. In one or more embodiments, in a neural network, parameter values may include weights and biases associated with the connection between neurons or nodes within layers of the network. In one or more embodiments, during training and/or pretraining of the machine-learning model, parameter values of the machine-learning model may be adjusted based on output of embeddings and correlated distances. In or more embodiments, processor 110 may be configured to minimize a loss function by adjusting parameter values of puncture machine-learning model 156 based on discrepancies between predicted outputs and actual outputs.

With continued reference to FIG. 1, puncture machine-learning model 156 may include any machine-learning model as described in this disclosure. In one or more embodiments, puncture machine-learning model 156 may be trained with puncture training data 160. In one or more embodiments, puncture training data 160 may include a plurality of treatment plans correlated to a plurality of puncture sites. In an embodiment, each treatment plan 164 may contain a correlated puncture site 152. For example, and without limitation, a treatment plan 164 containing a catheter selection with a low pliability metric may contain a correlated puncture site 152 that will require a medical professional to deform a catheter to reach area of interest 168. In some embodiments, a treatment plan 164 containing a catheter selection without steerability may include a path with minimal changes in direction or a puncture site 152 that is selected to allow for a path with minimal changes in direction. In one or more embodiments, puncture training data 160 may be generated based on historical data. In one or more embodiments, historical data may include previous medical procedures in which a perforation was made and the treatment plan 164 that was used. In one or more embodiments, historical data may include a puncture score indicating the success of each medical procedure. For example, and without limitation, the puncture score may include a range of 1-10, wherein a score of 1 may indicate that the medical procedure was not successful, and a puncture score of 10 may indicate that the medical procedure was successful. In one or more embodiments, the puncture score may be determined based on factors, such as but not limited to, additional and unintended perforations made, the ability to access the area of interest 168 from the puncture site 152 and/or the like. In one or more embodiments, the puncture scores may be used to train puncture machine-learning model 156, wherein for example, lower scores may indicate an unwanted output, and higher scores may indicate a desired output. In one or more embodiments, historical data may include factors, such as but not limited to, the amount of perforations made, the distance from the puncture site 152 to the area of interest 168, how long the procedure took and/or a medical professional's personal rating. In one or more embodiments, each factor within historical may be assigned a weighting and a correlated score. For example, and without limitation, perforation may be given a weighting of 0.33 wherein perforation may account for 33% fo the overall puncture score. Continuing, a lack of perforation may be given a 10, whereas each perforation may deduct 1 numerical value. Continuing, the non-limiting example, a medical procedure that resulted in three unintended perforations may contain a score of 7 and contribute to 33% of the overall puncture score. In one or more embodiments, puncture machine-learning model 156 may be iteratively trained wherein following each generation of puncture site 152 from puncture machine-learning model 156, a medical professional may provide feedback and/or puncture scores on the success of the medical procedure based on the puncture site 152. In one or more embodiments, feedback and/or puncture score may be used to adjust parameter values of puncture machine-learning model 156. In one or more embodiments, puncture scores may be used to rate puncture sites, wherein puncture machine-learning model 156 may be configured to output the puncture site 152 may be most closely related to the highest puncture score. In one or more embodiments, historical data may include a plurality of electronic health records associated with a plurality of individuals who previously received a medical procedure associated with accessing the left side of the heart. In one or more embodiments, historical data may be converted into numerical values and/or embodiments to reduce the dimensionality of historical data and thus increase an efficiency of puncture machine-learning model 156. In one or more embodiments, puncture training data 160 may include embeddings, vectors and/or numerical values representative of training data, wherein the embeddings, vectors and/or numerical values may be used to train puncture machine-learning model 156. In one or more embodiments, treatment plan 164 may be converted into an embedding and/or vector prior to input into puncture machine-learning model 156 to increase the efficiency of puncture machine-learning model 156. In one or more embodiments, puncture machine-learning model 156 may be trained to associate a particular selection of a catheter with an increase in probability of perforations. In one or more embodiments, puncture machine-learning model 156 may be trained to correlate pliability metrics to various areas of interest 168, wherein certain areas of interest may require a higher or lower pliability metric. In one or more embodiments, locating the puncture site 152 on the 3D organ model 137 as a function of a treatment plan 164 includes locating the puncture site 152 as a function of the area of interest 168 and a machine-learning model, such as puncture machine-learning model 156, wherein the machine-learning model is trained to correlate a plurality of areas of interest to a plurality of puncture sites. In one or more embodiments, processor 110 may be configured to compare generic 3D model 148 to 3D organ model 137, wherein processor 110 may be configured to convert values associated with puncture site 152 to be aligned within 3D organ model 137. In one or more embodiments, puncture machine-learning model 156 may be configured to output generic puncture sites, wherein generic puncture sites include puncture sites that are tailored to generic 3D model 148. In one or more embodiments, processor 110 may adjust values of generic puncture site to located puncture site 152. In an embodiment, training puncture machine-learning model 156 to output generic puncture sites may increase the accuracy of puncture machine-learning model 156 by limiting outputs to one organ size. In one or more embodiments, puncture sites within historical data may be converted to adjust for a generic 3D model 148, wherein historical data may then be used to train puncture machine-learning model 156. In one or more embodiments, puncture machine-learning model 156 may first be initialized by aggregating historical data, from a user, health database, 3^{rd} party and/or the like, then refined by iteratively training puncture machine-learning model 156 based on feedback provided from previous outputs. In one or more embodiments, processor 110 may increase computational efficiency by storing generic puncture site on a local memory 114 and using feedback associated with puncture site 152 to train puncture machine-learning model 156 with the outputted generic puncture site. In one or more embodiments, puncture machine-learning model 156 may be configured to minimize a distance of puncture site 152 and area of interest 168 by minimizing a loss function. In one or more embodiments, puncture machine-learning model 156 may be configured to minimize a loss function by locating puncture sites that are closest to area of interest 168 but will still result in a better puncture score. In one or more embodiments, puncture machine-learning model 156 may be trained using a reinforcement learning algorithm such as any reinforcement learning algorithm as described in this disclosure. In one or more embodiments, puncture machine-learning model 156 may be trained using historical data containing previous outputs of system 100 and correlated feedback associated with outputs of puncture machine-learning model 156. In one or more embodiments, the reinforcement algorithm may utilize procedure times and success to score outputs. This could include data collected from historical procedures. For example, and without limitation, longer procedure times and/or less successful procedures may be assigned a lower score and thus provide feedback to puncture machine-learning model 156 to generate outputs that result in faster procure times and/or more successful procedures.

With continued reference to FIG. 1, generating treatment guidance 150 includes identifying an access route 172 to area of interest 168. An “access route” for the purposes of this disclosure is a path that a catheter should follow when attempting to access area of interest. For example, and without limitation, access route 172 may include a path through pulmonary veins from puncture site 152 to area of interest 168. In one or more embodiments, access route 172 may include navigation through various portions of the heart to access area of interest 168. In one or more embodiments, processor 110 may identify various portions of the heart that obstruct puncture site 152 from area of interest 168, wherein access route 172 may identify a path around the obstructions. In one or more embodiments, access route 172 may include various perforations that need to be made, such as for example, perforations that would result in a catheter puncturing an obstruction to access area of interest 168. In one or more embodiments, access route 172 may include a distance and/or magnitude from puncture site 152 to area of interest 168. In one or more embodiments, magnitude may include vertical and horizontal measurements from puncture site 152 to area of interest 168. In one or more embodiments, distance may include the overall distance that a catheter travels from puncture site 152 to area of interest 168, such as for example, navigating nonlinear segments and/or the like. In one or more embodiments, access route 172 may include distance and/or magnitude. In one or more embodiments, access route 172 may include vectors that indicate magnitude and/or direction. In one or more embodiments, access route 172 may include obstructions that require navigation around and/or obstructions that require perforations to pass through and access area of interest 168. In one or more embodiments, access route 172 may include a particular path selected based on area of interest 168, catheter selection and/or pliability metric. In one or more embodiments, each access route 172 may differ based on the use of a differing catheter selection due to the ability of a catheter to deform or cause perforations.

With continued reference to FIG. 1, in some embodiments, puncture site 152 and access route 172 of treatment guidance 150 may be determined simultaneously. In some embodiments, processor 110 may determine puncture site 152 and access route 172 in parallel. In some embodiment, access route 172 may be first determined based on treatment plan 164 by identifying a path that would allow an instrument or medical device to reach the area of interest 168. Once this access route 172 has been established, processor 110 may locate a puncture site 152 by identifying a point on 3D organ model 137 where the access route 172 initiates, ensuring that the puncture site 152 accommodates the desired trajectory.

With continued reference to FIG. 1, in some embodiments, processor 110 may determine access route 172 and/or puncture site 152 as a function of required implantation angle. For the purposes of this disclosure, an “implantation angle” is an orientation or directional angle at which a medical device must be introduced into an anatomical structure to achieve proper positioning, deployment, and functional interaction with an area of interest. As a non-limiting example, implantation angle may be defined relative to a surface or axis of an area of interest (e.g., anatomical structure including LAA) into which the medical device (e.g., catheter, LAAC device, and the like) is to be implanted and may be measured with respect to anatomical planes or features of the organ. In some embodiments, implantation of a medical device (e.g., a left atrial appendage closure [LAAC] device) may impose specific requirements on the implantation angle at which the device must enter the target anatomical structure (e.g., LAA). For example, and without limitation, LAAC device may be delivered along a trajectory that allows precise alignment with the axis of the LA A ostium using implantation angle to ensure proper sealing, anchoring, and functional deployment. Deviation from the required implantation angle could result in inadequate sealing, device instability, or interference with adjacent cardiac structures, thereby compromising the safety and efficacy of the procedure.

With continued reference to FIG. 1, in some embodiments, processor 110 may reference a database, device specifications, or pre-trained machine-learning models that includes implantation angles required for specific devices and procedures. For example, and without limitation, when procedure type includes implantation of an LAAC device, processor 110 may retrieve or compute an implantation angle that corresponds to an axis normal or near normal to the LAA ostium plane. Processor 110 can then simulate or calculate the 3D orientation of the anatomical feature, such as the ostium, within 3D organ model 137, thereby defining the required implantation angle relative to the surrounding anatomical structures. In some embodiments, processor 110 may refine implantation angle by analyzing the 3D organ model 137, using segmentation, surface fitting, or geometric modeling techniques to more precisely characterize area of interest 168. In some embodiments, processor 110 may use a machine-learning model trained on procedural outcomes and anatomical variations to predict an optimal implantation angle for each individual patient based on morphology of area of interest 168. In some embodiments, processor 110 may determine implantation angle as a function of specific anatomical features within 3D organ model 137 that define the preferred orientation for device delivery. As a non-limiting example, implantation angle may refer to a centerline extending through an area of interest 168. For instance, processor 110 may generate a centerline that traverses the interior of left atrial appendage (LAA) from the ostium toward the distal tip, with the implantation angle being aligned along or derived from this centerline. In this embodiment, the implantation angle may be selected to be colinear or substantially colinear with the centerline. As another non-limiting example, processor 110 may determine implantation angle based on anatomical surfaces. For example, and without limitation, implantation angle may be selected to be parallel to a wall of the LAA. In such embodiments, processor 110 may analyze the surface geometry of LAA or area of interest 168 to identify planes or tangent vectors that characterize the wall orientation, and the implantation angle may be chosen accordingly to be parallel or nearly parallel to these identified anatomical surfaces.

With continued reference to FIG. 1, processor 110 may be configured to identify access route 172 based on puncture site 152. In one or more embodiments, each puncture site 152 and/or relative locations of puncture site 152 may include a correlated access route 172. In one or more embodiments, one puncture site 152 is determined, processor 110 may be configured to identify location of puncture site 152 and provide correlated access routes from puncture site 152. In one or more embodiments, various paths within 3D organ model 137 may be identified prior to generation of access route 172. In one or more embodiments, various paths may include section of a heart that may navigate using catheter. In one or more embodiments, all possible paths that may be navigated within a heart may be uploaded to a database. In one or more embodiments, processor 110 may be configured to identify puncture site 152 and area of interest 168 and aggregate paths to create access route 172 to area of interest 168. In one or more embodiments, processor 110 may use a machine-learning model such as any machine-learning model to identify access route 172 from puncture site 152 to area of interest 168. In one or more embodiments, machine-learning model may include an access machine-learning model 180. In one or more embodiments, access machine-learning model 180 may be configured to receive puncture site 152 and area of interest 168 and output access route 172. In one or more embodiments, access machine-learning model 180 may be trained with access training data 184. In one or more embodiments, access training data 184 may correlate a plurality of puncture sites and areas of interest to a plurality of access routes. In one or more embodiments, access machine-learning model 180 may be trained using historical data as described above. In one or more embodiments, historical data may include previous procedures in which a medical professional accessed the area of interest 168 from puncture site 152. In one or more embodiments, historical data may include access routes previously used in previous medical procedures. In one or more embodiments, access machine-learning model 180 may be trained with historical data. In one or more embodiments, access machine-learning model 180 may be configured to identify puncture sites and areas of interest within training data most closely related to puncture site 152 and area of interest 168 and provide correlated access routes. In one or more embodiments, access machine-learning model 180 may be configured to identify and/or generate access routes based on received puncture sites and/or areas of interests. In one or more embodiments, historical data may include a plurality of access paths. An “access path” for the purposes of this disclosure is a particular portion of a route taken from a puncture site 152 to an area of interest 168 during a medical procedure. For example, and without limitation, access path 176 may include navigation through a vein, navigation around an obstruction and/or the like. In one or more embodiments, historical data may include a plurality of access paths, wherein one or more access paths may define an access route 172. In one or more embodiments, access paths may be generated by segmenting access routes within historical data. In one or more embodiments, access routes may be segmented based on changes in position, orientation, direction, magnitude and/or the like. In one or more embodiments, access machine-learning model 180 may be trained using plurality of access paths, wherein access machine-learning model 180 may be configured to aggregate access paths to generate access route 172. In one or more embodiments, access machine-learning model 180 may be trained to minimize a loss function and generate access routes with the most minimal distance, change in direction and/or the like. In one or more embodiments, access machine-learning model 180 may be iteratively trained to generate access routes using access paths from historical data. In one or more embodiments, access machine-learning model 180 may be self-trained, wherein access machine-learning model 180 may be configured to generate a plurality of outputs and refine access machine-learning model 180 by identifying outputs with the least amount of distance and/or change in direction. In one or more embodiments, access paths may be specific to catheter selection and/or pliability metric. For example, and without limitation, an access path 176 requiring perforation may require a catheter capable of perforation. Similarly, an access path 176 requiring manipulation and/or bending of catheter may require a catheter selection that contains a pliability metric suitable for bending. In one or more embodiments, access machine-learning model 180 may output a generic path, wherein the generic path includes an access path 176 from generic 3D model 148. In one or more embodiments, processor 110 may then be configured to generic path to generate access route 172.

With continued reference to FIG. 1, processor 110 may be configured to determine an optimized access route from the puncture site to the area of interest, wherein the optimized access route is optimized for the procedure type. An “optimized access route” for the purposes of this disclosure is an access route that is suitable for an indicated procedure type within treatment plan. For example, and without limitation, a first access route may not be suitable for placement of a heart valve, whereas a second access route may be suitable for placement of a heart valve. In one or more embodiments, processor 110 may identify a plurality of access routes as described above and select one or more access routes based on procedural type. In one or more embodiments, various access paths may not be suitable for procedure types and/or cardiovascular devices, wherein processor 110 may select access routes that contain access routes with suitable access paths. In one or more embodiments, some access routes may be better suited than others for a particular procedure type. For example, and without limitation, a first access route generated, and a second access route generated may be suitable for a particular procedure type, while the first access route may be better suited. In one or more embodiments, access paths may include scoring indicating their suitability for each procedure type, wherein a particular access route generated may contain multiple scores, each indicating their suitability for the procedure type. In one or more embodiments, each access route may include procedure scores for each procedure type. A “procedure score” for the purposes of this disclosure is a numerical rating indicating a suitability of an access route for a particular procedure type. For example, and without limitation an access path may include a procedure score of 70 for heart valve placement and a procedure score of 90 for atrial mapping. In one or more embodiments, procedure scores may be generated similar to path scores. In one or more embodiments, procedure scores may include path scores that have been classified as a particular procedure type. For example, without limitation, failure within a particular procedure may lower a procedure score for said procedure but not lower the procedure score for other procedure types. In one or more embodiments, procedure scores may be generated based on deviations in linearity within access path, the amount of curves and/or edges and/or the like. In one or more embodiments, processor may be configured to aggregate procedure scores for each access route generated and determine an optimized access route containing the highest aggregated procedure score. In one or more embodiments, procedure types may be used to limit outputs of access machine-learning model, wherein access machine-learning model may receive procedure type as an input and ignore various access paths that are not suitable for the procedure type. In one or more embodiments, access machine-learning model may be configured to output a plurality of access routes and correlated procedure scores, wherein processor may select the access route with the most suitable procedure score. In one or more embodiments, plurality of access routes may exist wherein processor may be configured to determine an optimized access route based on the indicated procedure type.

With continued reference to FIG. 1, in one or more embodiments, determining optimized access route may include identifying a plurality of access paths located between the puncture site and the area of interest. In one or more embodiments, processor 110 may be configured to identify access paths that contain coordinates between puncture site and area of interest. In one or more embodiments, processor may be configured to calculate a procedure score for each access path of the plurality of access paths, wherein each access path includes more than one or more procedure scores and wherein at least one procedure score of the more than one procedure is correlated to the procedure type. In an embodiment, each access path may include a plurality of procedure scores wherein each procedure score contains a numerical rating of access path for a particular procedure type as described above. In one or more embodiments, optimized access route may be determined as a function of procedure score, wherein processor may select access route with the most suitable procedure score.

With continued reference to FIG. 1, procedure score may include an alignment metric between the cardiovascular device and the area of interest. An “alignment metric” for the purposes of this disclosure is an indication of as to whether a cardiovascular device will be properly aligned with an area of interest for proper implantation. For example, and without limitation, a catheter and cardiovascular device should enter the Left atrial appendage such that is aligned with the central axis of the appendage. If an angle between cardiovascular device and area of interest are off, the cardiovascular device may fail to fully seal an opening or sit flush against an LAA wall. In one or more embodiments, alignment metric may include an indication or probability as to whether cardiovascular device may be aligned or angled for proper insertion of cardiovascular device at or near area of interest. In one or more embodiments, a catheter may be used to navigate cardiovascular device through access route and/or access paths to transport cardiovascular device to area of interest. In one or more embodiments, each access route or path may require manipulation and/or rotation of catheter, thus resulting in rotation or manipulation of cardiovascular device. In one or more embodiments, procedure score may include alignment metric, wherein alignment metric may indicate the amount of rotation of manipulation of cardiovascular device during traversal of cardiovascular device through access paths. In one or more embodiments, processor 110 may be configured to optimize access path by selecting access paths that provide for a suitable alignment once catheter and/or cardiovascular device has reached area of interest. In one or more embodiments, alignment metric may include a change in rotation of cardiovascular device during traversal of access path, a change in direction of cardiovascular device and/or the like. In one or more embodiments, access routes may include aggregated alignment metrics, wherein aggregated alignment metrics may include alignment metrics from multiple access paths. In one or more embodiments, while a particular access path may indicate poor alignment, a subsequent access path may contain an alignment metric that realigns cardiovascular device through changes in rotation and/or manipulation. For example, and without limitation, a first access path may require a 4-degree angular rotation in a clockwise direction and a second access path may require a 4-degree angular rotation in a counterclockwise direction and thus effectively cancel out each other. In one or more embodiments, processor and/or access, machine-learning model may identify access routes based on aggregated alignment metrics, wherein the aggregated alignment metrics may indicate an overall change in angle, direction, manipulation and/or the like. In one or more embodiments, alignment metrics may be generated by access machine-learning model, input by a user and/or the like.

With continued reference to FIG. 1, generic 3D model 148 may be classified to contain various portions of the heart. For example, and without limitation, generic 3D model 148 may include labels indicating the aorta, the pulmonary veins and/or all other portions of the heart. In one or more embodiments, generic 3D model 148 may be stored and/or retrieved from database, wherein generic 3D model 148 may include classified portions of the heart. In one or more embodiments, portions of generic 3D model 148 may contain correlated coordinates indicating their location within a coordinate system. In one or more embodiments, generic 3D model 148 may include one or more organ constraints. An “organ constraint” for the purposes of this disclosure refers to a component of the heart that may obstruct a catheter from accessing an area of interest 168. In one or more embodiments, organ constraints may be labeled such as for example, “mitral valve.” In one or more embodiments, organ constraints may include coordinates indicating their location within a relative coordinate system of generic 3D model 148. In one or more embodiments, organ constraints may include and/or be included in cardiac feature datum. In one or more embodiments, organ constraints may include pliability metric. In one or more embodiments, pliability metric may indicate how susceptible organ constraint 188 is to a catheter. For example, and without limitation, an organ constraint 188 with high pliability metric may indicate that a pliable catheter may be required to prevent perforation of the organ constraint 188 when navigating through the heart. Similarly, organ constraint 188 may include a low pliability metric, wherein the low pliability metric may indicate that a catheter that is more rigid may be used. In one or more embodiments, a low pliability metric associated with organ constraint 188 may either indicate that organ constraint 188 is not susceptible to perforation and/or that organ constraint 188 can be perforated to allow for catheter to pass through. In one or more embodiments, organ constraints may include a pliability metric indicating their susceptibility to perforation and/or their effect when a perforation occurs. In one or more embodiments, organ constraints may include locations or ranges in which the organ constraints are located.

With continued reference to FIG. 1, processor 110 may segment generic 3D model 148 into a plurality of organ constraints, wherein each organ constraint comprises a differing portion of the heart, such as for example, a left chamber, a right chamber, a mitral valve and/or the like. In one or more embodiments, each organ constraint 188 may contain edges, boundaries or perimeter indicating the area defined by the organ constraint 188. In one or more embodiments, processor 110 may be configured to identify access routes by identifying paths, long edges or boundaries of each organ constraint 188. In one or more embodiments, boundaries may be identified within a coordinate system, wherein boundaries have already been identified prior to generation of access route 172, by an individual, operator, 3^{rd} party, machine vision system, and/or the like. In one or more embodiments, edges of organ constraints may be viewed as potential pathways for use in access route 172. In one or more embodiments, some organ constraints may allow for perforation, wherein a potential pathway may include the shortest distance through the organ constraint 188 that allows for perforation in relation to the area of interest 168. In one or more embodiments, processor 110 may determine identify a plurality of access paths, wherein each access path 176 includes a single edge of a single organ constraint 188. In one or more embodiments, processor 110 may be configured to identify a plurality of access paths within generic 3D model 148 by identifying all edges of all organ constraints. In one or more embodiments, plurality of access paths may be stored on a database. In one or more embodiments, at least one access path 176 of the plurality of access paths includes a portion of a boundary of at least one organ constraint 188 of the plurality of organ constraints.

With continued reference to FIG. 1, processor 110 may be configured to identify plurality of access paths by identifying plurality of organ constraints with 3D organ model 137 and/or generic 3D model 148. In one or more embodiments, processor 110 may identify plurality of access paths, based on edges or perimeter of organ constraints and generate an access route 172 from one or more of the plurality of access paths. In one or more embodiments, processor 110 may be configured to identify a vector between two points on organ model. In one or more embodiments, processor 110 may be configured to identify a vector between a point defined by puncture site 152 and a point defined by area of interest 168 within a coordinate system of generic 3D model 148. In one or more embodiments, processor 110 may be configured to select access path 176 located at or nearest to puncture site 152 and aggregate access paths from puncture site 152 to area of interest 168 to generate access route 172. In one or more embodiments, processor 110 may identify access route 172 by aggregating access paths that navigate from puncture site 152 to area of interest 168.

With continued reference to FIG. 1, in one or more embodiments, each access path 176 may include a path score. A “path score” for the purposes of this disclosure refers to a suitability of each access path 176 for navigating a catheter. For example, and without limitation, an access path 176 may include multiple turns or edges which may require excessive manipulation of catheter to navigate catheter through access path 176. In one or more embodiments, path score may include a numerical rating indicating the suitability of catheter to be navigated through. In one or more embodiments, path score may be determined based on angle deviations within access path 176, a requirement to rotate or bend catheter to navigate access path 176 and/or the like. In one or more embodiments, path score may indicate easiness in navigating a catheter through a particular access path 176. In one or more embodiments, path score may be tabulated based on the amount of angle deviations required, the amount of excessive flexion or rotation required and/or the like. In one or more embodiments, path score may further account for a severity of perforating an organ constraint 188 near path score. For example, and without limitation, path score may be low in instances in which access path 176 is a boundary of an artery, such that perforation of the artery may cause harm to the subject. In one or more embodiments, paths scores may be lower in instances in which edges or access paths are located near critical structures such as blood vessels. In one or more embodiments, path scores may be generated by a user, 3^{rd} party and/or the like. In one or more embodiments, each access path 176 may be initially given a path score, wherein access machine-learning model 180 may adjust path scores based on feedback received from an individual interacting with system. In one or more embodiments, each access path 176 be given a path score by processor 110, wherein processor may be configured to calculate path scores for each access path based on the linearity in the path, the distance to a critical structure and/or the like. In one or more embodiments, access path 176 may include a catheter manipulation score. A “catheter manipulation score” for the purposes of this disclosure is a numerical rating indicating the capability of a catheter to navigate an access path 176 based on its pliability metric. For example, and without limitation, a higher catheter manipulation score may indicate that an access route 172 requires a catheter capable of bending or deformation to navigate access route 172. In one or more embodiments, catheter manipulation rating may be used to identify if a particular catheter selection is suitable for navigation through access path 176. In one or more embodiments, catheter manipulation score may include a threshold indicating a maximum or minimum pliability metric needed in order navigate access path 176. For example, and without limitation, catheter manipulation score may indicate that a pliability metric associated with a steel catheter cannot be used, while a pliability rating associated with a silicone catheter capable of deformation may be used. In one or more embodiments, catheter manipulation score may indicate the maximum or minimum force, bending distance, pressure and/or the like that is needed to traverse access path 176. In one or more embodiments, catheter manipulation score may be used to identify if a particular catheter selection can be navigated through the access route 172. In one or more embodiments, processor 110 may be configured to identify access route 172 as a function of catheter manipulation score, wherein processor 110 may be configured to identify access paths that allow for selected catheter to be traversed. In one or more embodiments, access route 172 may be generated or identified based on catheter manipulation score, wherein access route 172 not adhering to the pliability metric may not be selected for use in access route 172. In one or more embodiments, access routes may be generated as a function of plurality of access paths.

With continued reference to FIG. 1, access machine-learning model 180 may be trained using plurality of access paths and/or path scores. In an embodiment, access machine-learning model 180 may be trained to generate an access route 172 containing the least amount of distance while also factoring aggregated path scores. In one or more embodiments, access machine-learning model 180 may use graph-based algorithms like Dijkstra's algorithm or A (A-star) to calculate the shortest or safest path between puncture site 152 and area of interest 168.

With continued reference to FIG. 1, In one or more embodiments, Dijkstra's algorithm and A* (A-star) are graph search algorithms used to find the most efficient path between two points in a network or map. In one or more embodiments, these algorithms can be used to find the most efficient route between puncture site 152 and area of interest 168. In one or more embodiments, algorithms may operate on a graph, where points (called nodes) represent possible positions or states, and the connections between them (edges) represent potential paths with associated costs, such as distance or risk. In the context of a transseptal puncture and catheter navigation, the heart and surrounding structures can be modeled as a graph, with anatomical landmarks as nodes and possible catheter paths as edges. A Dijkstra's algorithm may calculate and/or identify the shortest path from a starting node (e.g. puncture site) to a target node (e.g. area of interest) by exploring all possible paths (e.g. all possible access paths) and choosing the access paths with the least distance, the smallest path score and/or the least amount of turns or edges. In one or more embodiments, an A* algorithm uses a heuristic model to estimate the remaining distance from puncture site to area of interest. In one or more embodiments, the heuristic model may allow for the algorithm to guess the remaining distance from a particular point to area of interest and thus calculate or identify an access route. In one or more embodiments, algorithms described herein may be modified and/or configured to prioritize not only the shortest but also the safest path by incorporating clinical risk factors, such as proximity to critical structures, tissue thickness, or curvature of the path into the edge weights. In one or more embodiments, algorithms as described in this disclosure may be utilized path scores, procedural scores and/or the like in order to incorporate clinical risk factors. In one or more embodiments, the utilization of procedural scores and/or path scores may allow for algorithms described herein to find the safest access route simply by using numerical characters. In one or more embodiments, algorithms as described herein may be used in lieu of machine learning models to calculate and/or identify access route and/or optimized access route from puncture site to area of interest. In one or more embodiments, Dijkstra's algorithm and A* (A-star) algorithms may be used in lieu of machine learning models as described herein wherein said algorithms may provide predictable results. In one or more embodiments, Dijkstra's algorithm and A* (A-star) algorithms may provide predictable results, whereas machine learning models may generate inaccurate results. In one or more embodiments, Dijkstra's algorithm and A* (A-star) algorithms may provide for more transparent decision making, such that an individual may modify said algorithms to produce more predictable results. In one or more embodiments, Dijkstra's algorithm and A* (A-star) algorithms may lack large data sets in comparison to machine learning models and thus increase computational efficiency.

With continued reference to FIG. 1, in one or more embodiments, puncture sites and areas of interest may be represented as nodes within a coordinate system. In one or more embodiments, edges between the nodes may represent access paths, wherein access machine-learning model 180 may be configured to minimize a distance between puncture site 152 and area of interest 168 by identifying and/or selecting access paths suitable for minimization of distance. In one or more embodiments, access machine-learning model 180 may be configured to identify optimal access routes using access paths and correlated path scores. In one or more embodiments, access machine-learning model 180 may further be configured to disregard various access paths, when a pliability metric of a catheter does not allow for traversal through access paths. In one or more embodiments, access machine-learning model 180 may be configured to optimize aggregated path scores and distances for each access route 172. For example, and without limitation, access machine-learning model 180 may be iteratively trained to identify access routes with the highest aggregated path scores and the lowest or most optimal distance from puncture site 152 to area of interest 168.

With continued reference to FIG. 1, access machine-learning model 180 may be trained through reinforcement learning wherein access machine-learning model 180 may receive positive feedback for navigating closer to area of interest 168 and negative feedback for navigating further from area of interest 168. In one or more embodiments, reinforcement learning may further include positive feedback for access paths with high path scores and negative feedback for access paths with lower path scores. In one or more embodiments, access machine-learning model 180 may be trained through reinforcement learning to identify access routes with the most minimal distance while maintaining a high aggregated path score. In one or more embodiments, access machine-learning model 180 may be trained through reinforcement learning wherein access routes containing longer distances and/or lower path scores may be scored negatively using a scoring function and smaller distances and higher path scores may contribute positively to the scoring function. In one or more embodiments, reinforcement learning may allow for access machine-learning model to be iteratively trained by iteratively outputting access routes and using distances and/or correlated path scores to provide feedback without human intervention. In one or more embodiments, reinforcement learning may allow for access machine-learning model to be automatically trained by correlating distances and path scores to positive or negative feedback which may ultimately be used to retrain access machine-learning model. In one or more embodiments, access machine-learning model 180 may simulate the selection of a plurality of puncture sites correlated to a plurality of areas of interest and utilize feedback to generate access routes. In one or more embodiments, system 100 may utilize a machine-learning model such as access machine-learning model and/or puncture machine-learning model that has already been trained in any way as described in this disclosure. In one or more embodiments, historical data may include a plurality of optimal access routes, wherein optimal access routes are those conducted by medical professionals and labeled to be optimal. In one or more embodiments, access machine-learning model 180 may use plurality of optimal access routes to generate access routes for future iterations. In one or more embodiments, access machine-learning model 180 may include a 3 dimensional convolutional neural network and/or a graph neural network that maps as an input, the 3D organ model 137, the puncture point and the area of interest 168 and generates as an outputs, a sequence of 3D coordinates and/or access paths that form a safe route to the area of interest 168. In one or more embodiments, access machine-learning model 180 may first be trained using optimal access routes received from historical data, whereby access machine-learning model 180 may be iteratively trained through reinforcement learning to optimize routes and identify more efficient and/or safer access routes.

With continued reference to FIG. 1, in one or more embodiments, access machine-learning model 180 may be trained using generic 3D model 148, wherein outputs of access machine-learning model 180, such as access routes may be specific to generic 3D model 148. In one or more embodiments, processor 110 may be configured to compare 3D organ model 137 to generic 3D model 148 and adjust points within access route 172 to confirm generic 3D model 148. In one or more embodiments, use of generic 3D model 148 to output access route 172 may allow for more efficient training whereby a singular organ model is used to generate access routes.

With continued reference to FIG. 1, access machine-learning model 180, may receive as an input treatment plan 164, including catheter selection and pliability metric to identify access route 172. In one or more embodiments, access routes may differ based on the type of catheter used. In one or more embodiments, aggregation of path scores may not be as significant for various catheters due to their ability to deform and prevent damage to various anatomical structures. In one or more embodiments, access machine-learning model 180 may receive treatment plan 164 and select access route 172 based on treatment plan 164.

With continued reference to FIG. 1, access machine-learning model 180 may be configured to adjust path scores for each access path 176. In one or more embodiments, access machine-learning model 180 may use historical data to identify access paths that have been used within access routes. In one or more embodiments, the continuous or excessive use of a particular access path 176 may indicate that access path 176 is suitable for maneuvering through and thus receive a higher path score. In one or more embodiments, access machine-learning model 180 may receive feedback for generated access routes, wherein feedback may include the occurrence of a perforation. In one or more embodiments, processor 110 may identify the location of the perforation and lower path score for the access path 176 nearest to the perforation due to its perceived complexity due to mistakes made by medical professionals during procedures. In one or more embodiments, path scores may be iteratively updated, wherein access machine-learning model 180 may then be iteratively trained to adjust for updated path scores.

With continued reference to FIG. 1, processor may be configured to generate and/or calculate access routes by adding multiple access paths 176 to generate an access route 172. In one or more embodiments, processor 110 may be configured to identify a starting point of access route (e.g. puncture site) and an ending point of access route (e.g. area of interest), wherein processor 110 may be configured to identify the closest access path to the starting point and identify subsequent access paths that are closest to the previous access path. In one or more embodiments, processor may iteratively generate access routes by adding multiple access paths. In one or more embodiments, processor may be configured to compare already generated access routes to newly generated access routes and identify which access routes may be shortest and/or contain the highest level of linearity. In one or more embodiments, multiple access paths may be available for a particular access route. In one or more embodiments, processor 110 may be configured to generate an initial access route and modify and/or remove access paths within the initial access route until an ideal access route is created. In one or more embodiments, processor 110 may use linear functions to calculate distances and/or magnitudes for access routes by tabulating distances between beginning and points of each access path. In one or more embodiments, processor may select subsequent access paths to be used in access route if the initial location of the subsequent access path is similar to the end location of the previous access path. In an embodiment, a plurality of access paths within access route may create a continuous line from puncture site to area of interest.

With continued reference to FIG. 1, processor 110 is configured to generate an overlay 192 for 3D organ model 137 as a function of puncture site 152 and access route 172. An “overlay” for the purposes of this disclosure is a three-dimensional representation that is superimposed onto another Three-dimensional representation. For example, and without limitation, overlay 192 may include puncture site 152 and access route 172 overlayed onto 3D organ model 137. In one or more embodiments, overlay 192 may include a visualization of puncture site 152 and access route 172 on 3D organ model 137. In one or more embodiments, overlay 192 may provide a user, such as a medical professional, with graphical visualization of the portion of a subject's heart that requires puncture and the route to the area of interest 168. In one or more embodiments, puncture site 152 and access route 172 may be superimposed onto 3D organ model 137 by superimposing various location coordinates associated with puncture site 152, access route 172 and area of interest 168 onto 3D organ model 137.

With continued reference to FIG. 1, in one or more embodiments, creating overlay 192 may include generating a superimposed model. For the purposes of this disclosure, a “superimposed model” is a three-dimensional representation of puncture site 152 and access route 172 superimposed onto at least a 3D organ model 137. As a non-limiting example, superimposed model may include 3D representation of a perforation and a route to access area of interest 168 within a patient's heart. For the purposes of this disclosure, “superimpose” is the process of overlaying an image onto another image. In some embodiments, processor 110 may be further configured to determine a superimpose position for superimposed model. For the purpose of this disclosure, a “superimpose position” is a position that puncture site 152 and/or access route 172 can be superimposed on to a 3D cardiac model. As a non-limiting example, superimpose position may include a location of a puncture site 152 and access route 172 within 3D organ model 137. In an embodiment, superimpose position may include a position of superimposed model in a field coordinate system. As a non-limiting example, superimpose position may be obtained using a machine vision system. In one or more embodiments, overlay 192 may be displayed on display 108 for a user of system 100 to view. In an embodiment, overlay 192 may provide a pre-procedural map, akin to a GPS navigation system, which can be reviewed by the physician before the procedure. In one or more embodiments, overlay 192 may include a real-time superimposition of a transseptal needle and catheter position can be provided during the procedure, reducing reliance on fluoroscopy and enhancing procedural accuracy.

With continued reference to FIG. 1 system 100 may include a localization system configured to detect location data. Specifically, localization system may communicate with at least a probe and/or a catheter. As used in this disclosure, a “localization system” is a system that detects and determines the position of a medical device within a body or environment. In an embodiment, localization may produce signals, wherein the signals are generation as a function of at least a probe location (e.g., a location of ultrasound sensor), enabling precise tracking and navigation during medical procedures. In a non-limiting example, the purpose of at least a localization system may include enhancing the safety and efficacy of catheter-based interventions by providing critical spatial information. For purposes of this disclosure, “location data” refers to the information that specifies a medical device's spatial position relative to anatomical structures or reference points. Location data may be critical in ensuring that at least a probe is accurately positioned for diagnostic or therapeutic purposes. For example, in catheter-based interventions, location data might indicate that a catheter's position is within a blood vessel, relative to a lesion or stent deployment site.

In continued reference to FIG. 1, in an embodiment, location data may include spatial coordinates, orientation data, reference landmarks, temporal data, contextual information, and/or the like. Further, location data may be determined as a function of a plurality of position signals. As used in this disclosure, a “position signal” is a signal generated by localization system to determine the location of at least a probe within the body. In a non-limiting example, plurality of position signals may be derived from ultrasonic feedback, where localization system may use an array of transducers to emit ultrasonic waves and detect their reflections from a catheter. The resulting plurality of position signals may indicate the spatial coordinates of the catheter within the body and can be processed to adjust its positioning dynamically. In a non-limiting example, plurality of position signals may be represented as Cartesian coordinates (x, y, z) that define the location of at least a probe within a three-dimensional (3D) space. For instance, the localization system may generate plurality of position signals such as (12.5, 8.3, 4.7), indicating the exact spatial position of at least a probe relative to a defined origin point. In one or more embodiments, localization system may allow for real-time monitoring of a catheter as the catheter punctures puncture site and navigates to area of interest. Continuing, these coordinates may be continuously updated in real time to reflect the movement of at least a probe allowing precise tracking and guidance during a medical procedure. In another non-limiting example, plurality of position signals may include additional data such as orientation or angular displacement. For instance, plurality of position signals may take the form of a six-dimensional representation (x, y, z, roll, pitch, yaw), where the first three dimensions define the location and the latter three define the orientation of at least a probe. Which in some cases, may be integrated with a catheter; meaning that at least a probe positioning and orientation may be equivalent to catheter positioning and orientation. Continuing, this may enable localization system to provide more detailed guidance, particularly for tasks requiring precise alignment, such as positioning an ultrasonic transducer at a specific angle relative to a target region. In another non-limiting example, plurality of position signals may be expressed as polar coordinates (r, θ, φ), where “r” represents the radial distance from a reference point, “θ” the azimuthal angle, and “φ” the polar angle. Such a representation may be particularly useful in applications involving circular or spherical navigation paths, such as guiding a catheter around the walls of a heart chamber or within a curved blood vessel. Continuing, plurality of position signals may then be converted into actionable movement instructions for a requesting party, such as medical professional and/or a display device, to effect control on the placement and orientation of the catheter effectively. Precise placement and orientation may be necessary to capture a canonical view associated with a target region.

With continued reference to FIG. 1, in a non-limiting example, localization system may be consistent with one or more aspects of the localization system described in attorney docket number 1518-160USU1, U.S. patent application Ser. No. 18/764,853, filed on Jul. 5, 2024, titled “SYSTEM AND METHOD FOR LOCATING A MEDICAL DEVICE USING AN ELECTRICAL FIELD CREATION,” which is incorporated by reference herein in its entirety.

Referring now to FIG. 2A, an exemplary embodiment of a graphical visualization 200a is illustrated. In one or more embodiments, graphical visualization 200a is displayed on display device 204. In one or more embodiments, display device 204 may include any user device and/or display as described in this disclosure. In one or more embodiments, graphical visualization 200a includes 3D organ model 208 and overlay. In one or more embodiments, overlay may depict puncture site 212 and access route 216 from puncture site 212 to area of interest 220. In one or more embodiments, area of interest 220 may include a location in which treatment is sought. In one or more embodiments, access route may provide a medical professional with guidance on how to access area of interest 220. In one or more embodiments, 3D organ model 208 may include a three-dimensional digital representation of a subject's heart. In one or more embodiments, the three-dimensional representation may allow for a medical professional to analyze a subject's heart through a display device 204. In one or more embodiments, overlay may visualize a procedure in which punctures are made in the heart to access the left atrium. In one or more embodiments, overlay may provide a visualization for a procedure such as a transseptal puncture. In one or more embodiments, graphical visualization 200a may further include a real-time overlay of a transseptal needle and a catheter position during a procedure, reducing reliance on fluoroscopy and enhancing procedural accuracy. In one or more embodiments, graphical visualization 200a may be generated in any way as described in reference to at least FIG. 1. In one or more embodiments, graphical visualization 200a may eliminate unnecessary catheter manipulations, preventing cases where standard puncture sites lead to lengthy adjustments. In one or more embodiments, graphical visualization 200a may reduce the risk of complications such as thrombus formation and embolization, and improved procedural outcomes by ensuring consistent and optimized device placement, improving success rates for interventions. In one or more embodiments, a system such as any system as described in this disclosure may minimize fluoroscopy dependence by integrating real-time ultrasound visualization. In one or more embodiments, the system has the potential to reduce radiation exposure to both the patient and the physician. In one or more embodiments, graphical visualization 200a can be incorporated into existing imaging platforms used in catheterization labs. In one or more embodiments, a combination of machine-learning and geometric modeling may be used to refine site selection and path prediction based on procedural outcomes. In one or more embodiments, graphical visualization 200a may provide a novel, data-driven approach to transseptal puncture planning and execution, significantly improving procedural efficiency and patient safety. By leveraging 3D ultrasound imaging and computational algorithms, the system enables precise, patient-specific transseptal access, paving the way for safer and more efficient left-heart interventions.

Referring now to FIG. 2B, an exemplary embodiment of a graphical visualization 200b is illustrated. In one or more embodiments, graphical visualization 200b includes 3D organ model 208 and overlay. In one or more embodiments, overlay may depict puncture site 212 and access route 216 to area of interest 220. In one or more embodiments, area of interest 220 may include a location in which treatment is sought. As a non-limiting example, area of interest 220 may include left atrium appendage (LAA) 224, and the like. In one or more embodiments, access route 216 may provide a medical professional with guidance on how to access area of interest 220. In one or more embodiments, overlay may visualize a procedure in which punctures are made in the septum to access the left atrium appendage 224. In one or more embodiments, overlay may provide a visualization for a procedure such as a transseptal puncture. In one or more embodiments, graphical visualization 200a may further include a real-time overlay of a transseptal needle and a catheter position during a procedure, reducing reliance on fluoroscopy and enhancing procedural accuracy. In some embodiments, the implantation of a medical device (e.g., a left atrial appendage closure (LAAC) device 228) imposes specific requirements on implantation angle 232 at which the device must enter the target anatomical structure (e.g., LAA 224). In some embodiments, processor may determine optimal access route 216 and/or puncture site 212 as a function of required implantation angle 232. For example, and without limitation, LAAC device 228 may be delivered along a trajectory that allows precise alignment with the axis of the LAA 224 ostium using implantation angle 232 to ensure proper sealing, anchoring, and functional deployment. Deviation from the required implantation angle could result in inadequate sealing, device instability, or interference with adjacent cardiac structures, thereby compromising the safety and efficacy of the procedure.

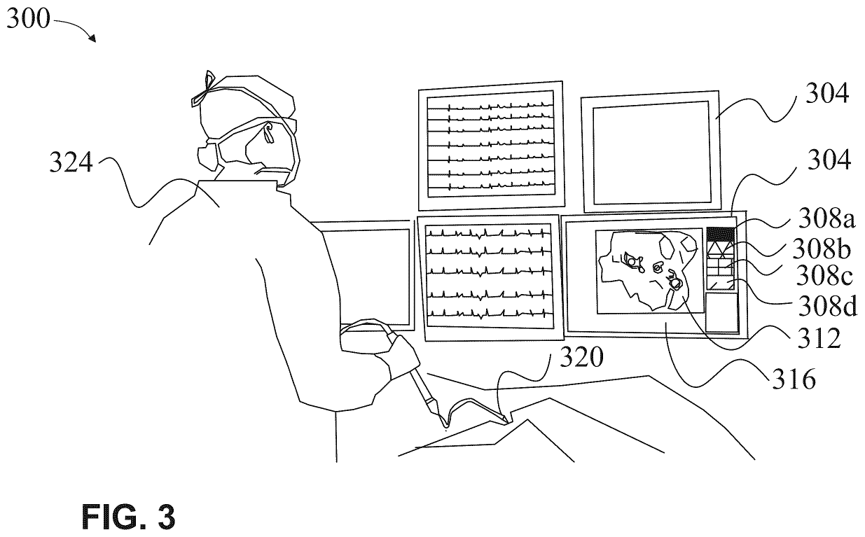

Referring now to FIG. 3, an illustration 300 of a downstream device displaying a color scale associated with a score during a procedure. In one or more embodiments, procedure may include any procedure such as but not limited to, an ablation procedure, a transseptal puncture, and/or any other procedure as described in this disclosure. In one or more embodiments, downstream device 304 may be configured to perform one or more processes as described in reference to at least FIGS. 1-2. In one or more embodiments, downstream device may be configured to depict puncture sites, 3D organ model, access routes, overlays and/or the like. In one or more embodiments, during a procedure, a medical professional may view illustration 300 and identify a location of catheter and an access route. In an embodiment, the illustration 300 may include a downstream device 304, a color scale first value (CSFV) 308a, a color scale second value (CSSV) 308b, a color scale third value (CSTV) 308c, a color scale fourth value (CSFV) 308d, a visual element 312, a graphical user interface (GUI) 316, an ablation procedure 320, and a surgeon 324. In an embodiment, the downstream device 304 may display various data and visual elements related to the ablation procedure 320. In an embodiment, the downstream device 304 may be configured to present information to surgeon 324 through GUI 316. In an embodiment, GUI 316 may include multiple screens showing different types of data and visualizations. In an embodiment, the color scale first value (CSFV) 308a represents the initial value on the color scale used to indicate tissue susceptibility. In an embodiment, the CSFV 308a is part of the color scale displayed on the downstream device 304. In an embodiment, the color scale second value (CSSV) 308b may follow the CSFV 308a and represents the next value on the color scale. In an embodiment, the color scale third value (CSTV) 308c may follow the CSSV 308b and represents the subsequent value on the color scale. In an embodiment, the color scale fourth value (CSFV) 308d may follow the CSTV 308c and represents the final value on the color scale. In an embodiment, the visual element 312 may be displayed on the downstream device 304 and is associated with the score generated during the ablation procedure 320. In an embodiment, the visual element 312 may utilize the color scale to provide a visual representation of the tissue susceptibility score. GUI 316 integrates the visual element 312 and the color scale to assist the surgeon 324 in monitoring the ablation procedure 320. In an embodiment, the ablation procedure 320 may be monitored by surgeon 324 using the downstream device 304. In an embodiment, surgeon 324 may interact with the GUI 316 to assess the tissue susceptibility score and make informed decisions during the ablation procedure 320. In an embodiment, the illustration 300 may provide a comprehensive view of the interaction between the surgeon 324, the downstream device 304, and the visual elements displayed during the ablation procedure 320.

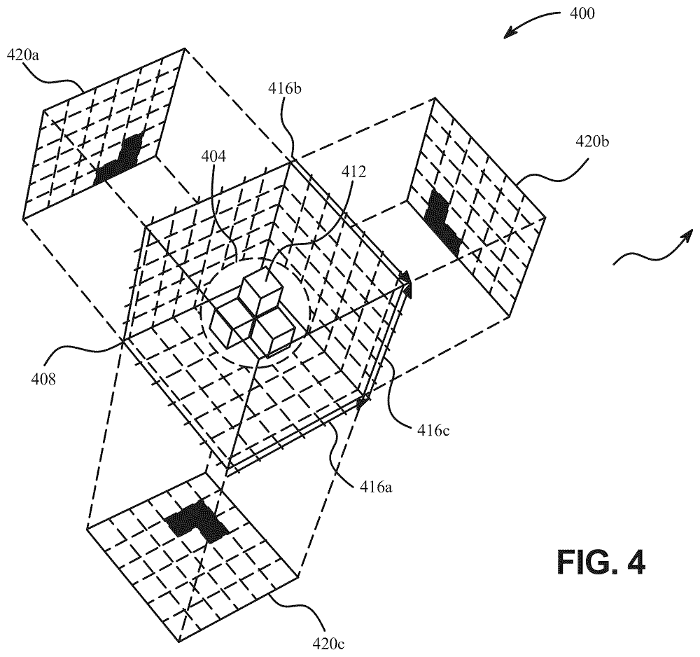

Now referring to FIG. 4, an exemplary embodiment of a 3D VOR 400 is illustrated. 3D VOR 400 may be used to represent 3D object 404. In an embodiment, 3D VOR 400 may divide a 3D space 408 into a grid of one or more cubic units e.g., voxels 412, wherein each voxel 412 represents a specific volume within 3D space 408. In a non-limiting example, 3D objects 404 may include a structure pertaining to a subject.

Still referring to FIG. 4, in some cases, each voxel 412 may act as a basic building block. In a non-limiting example, each voxel 412 may be configured to represent a discrete portion of 3D space 408. In an embodiment, each voxel 412 may include a presence indicator as described above with reference to FIG. 1, which denotes whether the voxel is occupied or unoccupied. In such embodiment, the binary or continuous value may allow 3D VOR 400 to map the presence or absence of material within each voxel 412, creating a granular representation of 3D object 404.

With continued reference to FIG. 4, in some cases, the resolution of 3D VOR 400 may be determined by the size and number of voxels within the grid. In a non-limiting example, smaller voxel may provide a higher resolution, capturing finer details, while larger voxels offer a more generalized representation.

Still referring to FIG. 4, in an embodiment, voxels 412 may be arranged in a regular pattern along three axis 416a-c (416a, 416b, 416c), each pointing a distinct direction. In a non-limiting example, voxels 412 may be arranged along x, y, and z axes, wherein such arrangement may facilitate efficient manipulation and rendering of the 3D object 404. In some cases, cardiac feature datums 420a-c (420a, 420b, 420c) such as, without limitation, edges, surfaces, textures, and any other cardiac feature datums as described above with reference to FIG. 1, may be extracted from 3D VOR 400 by analyzing the relationships and patterns between neighboring voxels.

Now referring to FIG. 5, a schematic of an exemplary transesophageal echocardiogram (TEE) procedure 500 is shown. In some cases, TEE procedure 500 may be performed during another procedure, for instance heart surgery. According to some embodiments, patient 504 has an endoscope 508, with an ultrasonic transducer 512, inserted into his esophagus 516. As one's esophagus 516 is proximal to one's heart 520, ultrasonic transducer 512 may generate echocardiograms.

Still referring to FIG. 5, in some embodiments, transesophageal echocardiography (TEE) may provide superior imaging quality than intracardiac echocardiography (ICE), as larger ultrasound transducers 512 may be placed within the esophagus 516 than within heart 520. In some cases, ultrasound transducers may be substantially miniaturized to fit within heart 520, as in ICE catheters. As esophagus 516 may be proximal to heart 520, TEE may provide a clear image of various heart structures without needing vascular access (as commonly required by ICE). Additionally, TEE may be performed without obstructing patient's 504 ribcage and intermediary tissues (as commonly required by transthoracic echocardiography [TTE]). In some cases, TEE images may also provide information associated with angle of acquisition. Angle of acquisition may be an angle of TEE probe with respect to esophagus 516 (e.g., esophageal axis).

Still referring to FIG. 5, in some embodiments, TEE echocardiogram data, including images showing heart structures and, in some cases, angle of acquisition, may be used as input to any machine-learning process described in this application, for instance with reference to FIGS. 1-4. For instance, TEE echocardiogram data may be used to reconstruct 3D heart models. In some cases, TEE echocardiogram data may be input into a machine-learning model that outputs a 3D heart model (e.g., 3D mesh model and/or statistical shape model).

Still referring to FIG. 5, in some embodiments, TEE may be a preferred imaging modality for structural heart interventions, such as without limitation left atrial appendage occlusion (LAOO), a stent, or a valve. In some cases, technology and improvements described in this disclosure permit creation and/or modification of a 3D heart mesh from TEE data to aid in planning implant size selection, as well as to guide implantation procedures. In some cases, virtual placement of a 3D model of a candidate implant (such as without limitation LAAO device) can be simulated on a 3D heart model generated by any method described in this disclosure. This novel and improved functionality may validate appropriate size and placement of implants within heart 520, as well as other organs within body of patient 504. For example, in the context of electrophysiology procedures, TEE procedure 500 can be used to create heart anatomical models that can be used as reference for electroanatomic mapping, and guidance of ablation catheters for atrial fibrillation treatment procedures (such as without limitation, puncturing septum for Cardiovascular device placement).

Still referring to FIG. 5, in some embodiments, applications described with reference to TEE procedure 500 above can be extended for use with TTE and point of care ultrasound (POCUS). In some cases, both TTE and POCUS may acquire ultrasound images of chest/surface of patient 504. In some cases, TTE and POCUS data may be used as an input (and/or training data) for any machine-learning process described in this disclosure, for instance with reference to FIGS. 1-4 and 6-10. In some cases, use of TTE and/or POCUS data (in machine-learning processes described in this disclosure) may require adjustment in ultrasound acquisition parameters and positions to acquire a sufficient number of frames for 3D reconstruction. In some cases, TTE and POCUS may offer improved accessibility (with POCUS being portable/mobile as well) and non-invasive 3D heart modeling, often without anesthesia or sedation, compared to catheterized 3D heart modeling commonly performed today for electroanatomical mapping and ablation procedures.

Referring now to FIG. 6, an exemplary embodiment of a machine-learning module 600 that may perform one or more machine-learning processes as described in this disclosure is illustrated. Machine-learning module may perform determinations, classification, and/or analysis steps, methods, processes, or the like as described in this disclosure using machine-learning processes. A “machine-learning process,” as used in this disclosure, is a process that automatedly uses training data 604 to generate an algorithm instantiated in hardware or software logic, data structures, and/or functions that will be performed by a computing device/module to produce outputs 608 given data provided as inputs 612; this is in contrast to a non-machine-learning software program where the commands to be executed are determined in advance by a user and written in a programming language.

Still referring to FIG. 6, “training data,” as used herein, is data containing correlations that a machine-learning process may use to model relationships between two or more categories of data elements. For instance, and without limitation, training data 604 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 604 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 604 according to various correlations; correlations may indicate causative and/or predictive links between categories of data elements, which may be modeled as relationships such as mathematical relationships by machine-learning processes as described in further detail below. Training data 604 may be formatted and/or organized by categories of data elements, for instance by associating data elements with one or more descriptors corresponding to categories of data elements. As a non-limiting example, training data 604 may include data entered in standardized forms by persons or processes, such that entry of a given data element in a given field in a form may be mapped to one or more descriptors of categories. Elements in training data 604 may be linked to descriptors of categories by tags, tokens, or other data elements; for instance, and without limitation, training data 604 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively, or additionally, and continuing to refer to FIG. 6, training data 604 may include one or more elements that are not categorized; that is, training data 604 may not be formatted or contain descriptors for some elements of data. Machine-learning algorithms and/or other processes may sort training data 604 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data to be made applicable for two or more distinct machine-learning algorithms as described in further detail below. Training data 604 used by machine-learning module 600 may correlate any input data as described in this disclosure to any output data as described in this disclosure. As a non-limiting illustrative example, input data may include 3D organ model, puncture sites, and/or the like and outputs may include puncture sites, access routes and/or the like.

Further referring to FIG. 6, training data may be filtered, sorted, and/or selected using one or more supervised and/or unsupervised machine-learning processes and/or models as described in further detail below; such models may include without limitation a training data classifier 616. Training data classifier 616 may include a “classifier,” which as used in this disclosure is a machine-learning model as defined below, such as a data structure representing and/or using a mathematical model, neural net, or program generated by a machine-learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 600 may generate a classifier using a classification algorithm, defined as a process whereby a computing device and/or any module and/or component operating thereon derives a classifier from training data 604. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 616 may classify elements of training data to a patient cohort related to patient's age, gender, medical experience, medical record, and the like.

Still referring to FIG. 6, Computing device may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)=P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device may then calculate a likelihood table by calculating probabilities of different data entries and classification labels. Computing device may utilize a naïve Bayes equation to calculate posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 6, computing device may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 6, generating k-nearest neighbors algorithm may generate a first vector output containing a data entry cluster, generating a second vector output containing an input data, and calculating the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute/as derived using a Pythagorean norm:

$$l = \sqrt{{\sum }_{i = 0}^{n} {a}_{i}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 6, training examples for use as training data may be selected from a population of potential examples according to cohorts relevant to an analytical problem to be solved, a classification task, or the like. Alternatively, or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data analyzed are represented by more training examples than values that are encountered less frequently. Alternatively, or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 6, computer, processor, and/or module may be configured to preprocess training data. “Preprocessing” training data, as used in this disclosure, is transforming training data from raw form to a format that can be used for training a machine-learning model. Preprocessing may include sanitizing, feature selection, feature scaling, data augmentation and the like.

Still referring to FIG. 6, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively, or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 6, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity, and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators to take advantage of several image statistics as texture descriptors to compute a focus level. Blur detection may be performed by using discrete cosine transform (DCT) coefficients to compute a focus level of an image from its frequency content.

Continuing to refer to FIG. 6, computing device, processor, and/or module may be configured to precondition one or more training examples. For instance, and without limitation, where a machine-learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however the desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine-learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been down sampled to smaller numbers of pixels, and a neural network or other machine-learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively, or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 6, computing device, processor, and/or module may down-sample elements of a training example to a desired lower number of data elements. As a non-limiting example, a high pixel count image may have 256 pixels, however the desired number of pixels may be 128. Processor may down-sample the high pixel count image to convert the 256 pixels into 128 pixels. In some embodiments, processor may be configured to perform down sampling on data. Down sampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean upside-effects of compression.

Further referring to FIG. 6, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 6, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset Xₘₐₓ:

$${X}_{n e w} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{n e w} = \frac{X - {X}_{m e a n}}{{X}_{max} - {X}_{min}} .$$

Feature scaling may include standardization, where a difference between X and Xₘₑₐₙ is divided by a standard deviation σ of a set or subset of values:

$${X}_{n e w} = \frac{X - {X}_{m e a n}}{σ} .$$

Scaling may be performed using a median value of a set or subset X_{median} and/or interquartile range (IQR), which represents the difference between the 25th percentile value and the 50th percentile value (or closest values thereto by a rounding protocol), such as:

$${X}_{n e w} = \frac{X - {X}_{m e d i a n}}{I Q R} .$$

Persons skilled in the art, upon reviewing the entirety of the disclosure, will be aware of various alternative or additional approaches that may be used for feature scaling.

Further referring to FIG. 6, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 6, machine-learning module 600 may be configured to perform a lazy-learning process 620 and/or protocol, which may alternatively be referred to as a “lazy loading” or “call-when-needed” process and/or protocol, may be a process whereby machine-learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 604. Heuristic may include selecting some number of highest-ranking associations and/or training data 604 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively, or additionally, and with continued reference to FIG. 6, machine-learning processes as described in this disclosure may be used to generate machine-learning models 624. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 624 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 624 may be generated by creating an artificial neural network, such as a convolutional neural network comprising an input layer of nodes, one or more intermediate layers, and an output layer of nodes. Connections between nodes may be created via the process of “training” the network, in which elements from a training data 604 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 6, machine-learning algorithms may include at least a supervised machine-learning process 628. At least a supervised machine-learning process 628, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include inputs such as 3D organ model, Punctures sites and/or the like and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 604. Persons skilled in the art, upon reviewing the entirety of this disclosure, will be aware of various possible variations of at least a supervised machine-learning process 628 that may be used to determine relation between inputs and outputs. Supervised machine-learning processes may include classification algorithms as defined above.

With further reference to FIG. 6, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including, without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively, or additionally, one or more errors and/or error function values evaluated in training iterations may be compared to a threshold.

Still referring to FIG. 6, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 6, machine-learning processes may include at least an unsupervised machine-learning processes 632. A n unsupervised machine-learning process, as used herein, is a process that derives inferences in datasets without regard to labels; as a result, an unsupervised machine-learning process may be free to discover any structure, relationship, and/or correlation provided in the data. Unsupervised processes 632 may not require a response variable; unsupervised processes 632 may be used to find interesting patterns and/or inferences between variables, to determine a degree of correlation between two or more variables, or the like.

Still referring to FIG. 6, machine-learning module 600 may be designed and configured to create a machine-learning model 624 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 6, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including, without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 6, a machine-learning model and/or process may be deployed or instantiated by incorporation into a program, system, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 6, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 6, retraining and/or additional training may be performed using any process for training described above, using any current or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, system, and/or method described in this disclosure; such examples may be modified and/or labeled according to user feedback or other processes to indicate desired results, and/or may have actual or measured results from a process being modeled and/or predicted by system, module, machine-learning model or algorithm, system, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 6, one or more processes or algorithms described above may be performed by at least a dedicated hardware unit 636. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 636 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 636 may include, without limitation, graphical processing units (GPUs), dedicated signal processing modules, FPGA or other reconfigurable hardware that has been configured to instantiate parallel processing units for one or more specific tasks, or the like, A computing device, processor, system, or module may be configured to instruct one or more dedicated hardware units 636 to perform one or more operations described herein, such as evaluation of model and/or algorithm outputs, one-time or iterative updates to parameters, coefficients, weights, and/or biases, and/or any other operations such as vector and/or matrix operations as described in this disclosure.

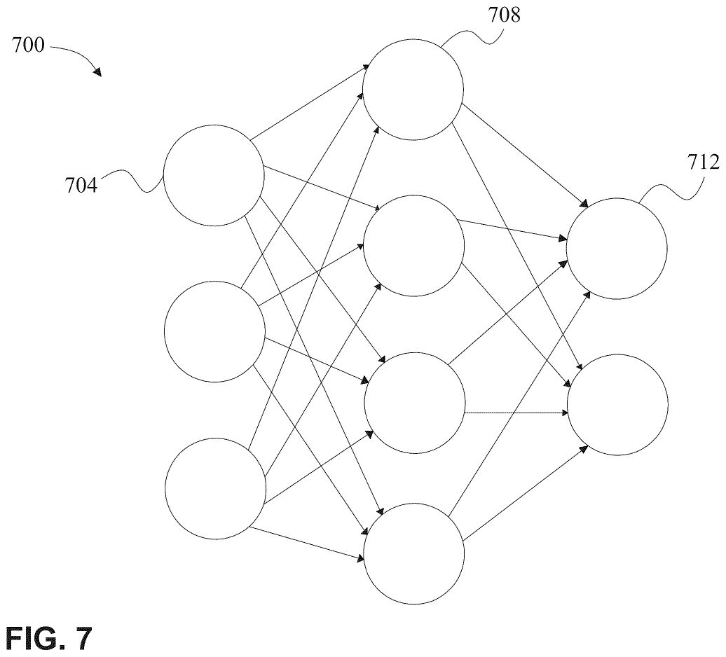

Referring now to FIG. 7, an exemplary embodiment of neural network 700 is illustrated. A neural network 700 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 704, one or more intermediate layers 708, and an output layer of nodes 712. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

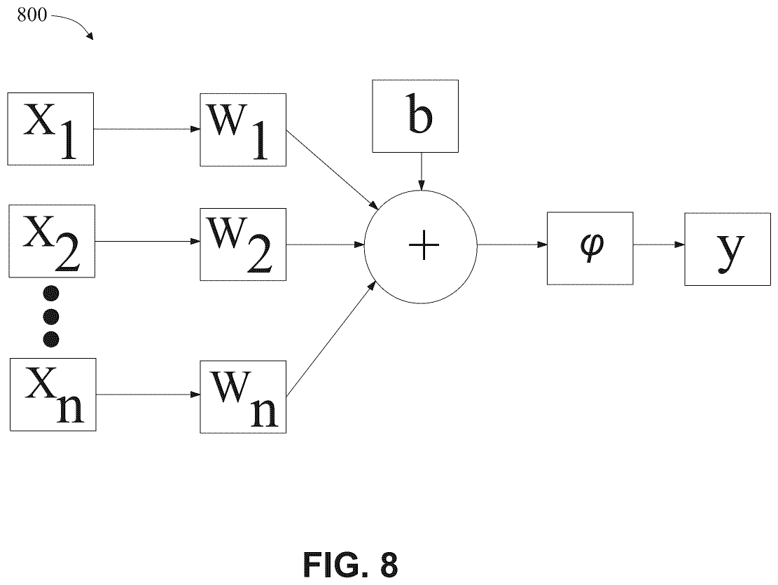

Referring now to FIG. 8, an exemplary embodiment of a node 800 of a neural network is illustrated. A node may include, without limitation, a plurality of inputs xᵢ that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form f(x)=1/1-e^{-x} given input x, a tanh (hyperbolic tangent) function, of the form

$$\frac{{e}^{x} - {e}^{- x}}{{e}^{x} + {e}^{- x}} ,$$

a tanh derivative function such as f(x)=tanh²(x), a rectified linear unit function such as f(x)=max(0, x), a “leaky” and/or “parametric” rectified linear unit function such as f(x)=max(ax, x) for some a, an exponential linear units function such as

$$f ( x ) = \{ \begin{matrix}x for x \geq 0 \\ α ( {e}^{x} - 1 ) for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as f(x)=x*sigmoid(x), a Gaussian error linear unit function such as f(x)=a(1+tanh(\sqrt{2/π}(x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs xᵢ that may be used as activation functions. As a non-limiting and illustrative example, node may perform a weighted sum of inputs using weights wᵢ that are multiplied by respective inputs xᵢ. Additionally, or alternatively, a bias b may be added to the weighted sum of the inputs such that an offset is added to each unit in the neural network layer that is independent of the input to the layer. The weighted sum may then be input into a function p, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

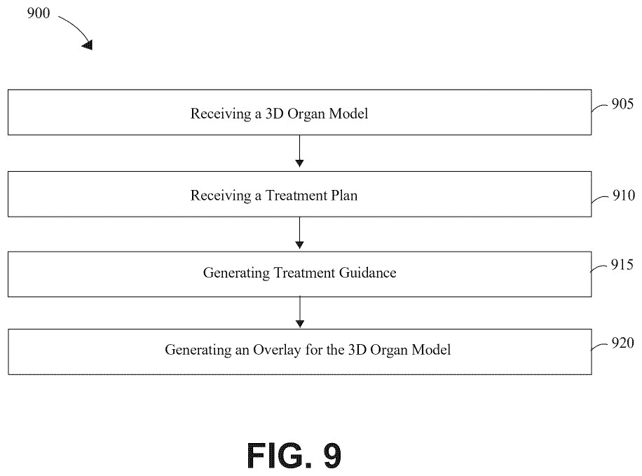

Referring now to FIG. 9, a method 900 for transseptal puncture site selection is described. At step 905, method 900 includes receiving, by at least a processor, a 3D organ model pertaining to an organ of a subject. This may be implemented with reference to FIGS. 1-9 and without limitation.

With continued reference to FIG. 9, at step 910, method 900 includes receiving, by the at least a processor, a treatment plan pertaining to the subject, wherein the treatment plan includes at least an area of interest and a procedure type. This may be implemented with reference to FIGS. 1-9 and without limitation.

With continued reference to FIG. 9, at step 915, method 900 includes generating treatment guidance as a function of a treatment plan. Generating the treatment guidance includes locating, by the at least a processor, a puncture site on the 3D organ model as a function of the treatment plan, wherein locating the puncture site includes determining the puncture site using a puncture machine-learning model, wherein the puncture machine-learning model is configured to receive the treatment plan as input and output a puncture site. Generating the treatment guidance includes determining, by the at least a processor, an optimized access route to the area of interest, wherein the optimized access route is optimized for the procedure type. This may be implemented with reference to FIGS. 1-9 and without limitation.

With continued reference to FIG. 9, at step 920, method 900 includes generating, by the at least a processor, an overlay for the 3D organ model as a function of the puncture site and the access route. In one or more embodiments, method 900 further includes receiving, by at least a transesophageal echocardiogram system, one or more ultrasound images of the subject wherein the transesophageal echocardiogram system includes least an ultrasound sensor configured to be located within an esophagus of the subject and capture the one or more ultrasound images of the subject. In one or more embodiments, receiving the 3D organ model includes generating the 3D organ model as a function of the one or more ultrasound images. In one or more embodiments, the method further includes receiving, by at least an intracardiac echocardiography system, one or more ultrasound images of the subject, wherein the intracardiac echocardiography system includes at least an ultrasound sensor configured to be inserted into a vein of the subject and capture one or more ultrasound images of a heart of the subject. In one or more embodiments, receiving the 3D organ model includes generating the 3D organ model as a function of the one or more ultrasound images. In one or more embodiments, the treatment plan includes a catheter selection. In one or more embodiments, the catheter selection includes a pliability metric and/or a steerability metric. In one or more embodiments, the puncture site includes a location coordinate on the 3D organ model. In one or more embodiments, locating the puncture site on the 3D organ model as a function of a treatment plan includes locating the puncture site as a function of the area of interest and a machine-learning model, wherein the machine-learning model is trained with historical data to correlate a plurality of areas of interest to a plurality of puncture sites. In one or more embodiments, identifying the access route to the area of interest includes receiving a plurality of organ constraints within the 3D organ model, identifying a plurality of access paths as a function of the plurality of organ constraints, wherein at least one access path of the plurality of access paths includes a portion of a boundary of at least one organ constraint of the plurality of organ constraints and identifying the access route as a function of the plurality of access paths. In one or more embodiments, identifying the access route as a function of the plurality of access paths includes receiving a path score for each access path of the plurality of access paths, wherein the path score includes at least a catheter manipulation score and identifying the access route as a function of the catheter manipulation score. In one or more embodiments, the plurality of organ constraints includes a pliability metric. This may be implemented with reference to FIGS. 1-9 and without limitation.

With continued reference to FIG. 9, in one or more embodiments, the method further includes receiving, by at least an ultrasound imaging device, one or more ultrasound images of the subject wherein the ultrasound imaging devices includes least an ultrasound sensor configured to be located within an esophagus of the subject and capture the one or more ultrasound images of the subject and receiving the 3D organ model includes generating the 3D organ model as a function of the one or more ultrasound images. In one or more embodiments, generating the 3D organ model as a function of the one or more ultrasound images includes generating a set of shape parameters representing a shape of the organ as a function of the one or more of ultrasound images and a shape identification model trained on a training dataset including historical ultrasonic images correlated with historical computed tomography scan data and generating the 3D organ model of the organ based on the set of shape parameters. In one or more embodiments, the treatment plan includes a catheter selection. In one or more embodiments, the catheter selection includes a pliability metric. In one or more embodiments, the puncture machine-learning model has been trained with historical data to correlate a plurality of areas of interest to a plurality of puncture sites. In one or more embodiments, identifying the access route to the area of interest includes receiving a plurality of organ constraints within the 3D organ model, identifying a plurality of access paths as a function of the plurality of organ constraints, wherein at least one access path of the plurality of access paths includes a portion of a boundary of at least one organ constraint of the plurality of organ constraints and identifying the access route as a function of the plurality of access paths. In one or more embodiments, determining the optimized access route includes identifying a plurality of access paths located between the puncture site and the area of interest, receiving a path score for each access path of the plurality of access paths, wherein the path score includes at least a catheter manipulation score and determining the optimized access route as a function of the catheter manipulation score. In one or more embodiments, the procedure type includes a selection of a cardiovascular device to be navigated to the area of interest and determining the optimized access route includes identifying a plurality of access paths located between the puncture site and the area of interest, calculating a procedure score for each access path of the plurality of access paths, wherein each access path includes more than one or more procedure scores and wherein at least one procedure score of the more than one procedure is correlated to the procedure type and determining the optimized access route as a function of the path score. In one or more embodiments, the procedure score includes an alignment metric between the cardiovascular device and the area of interest. This may be implemented with reference to FIGS. 1-9 and without limitation.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

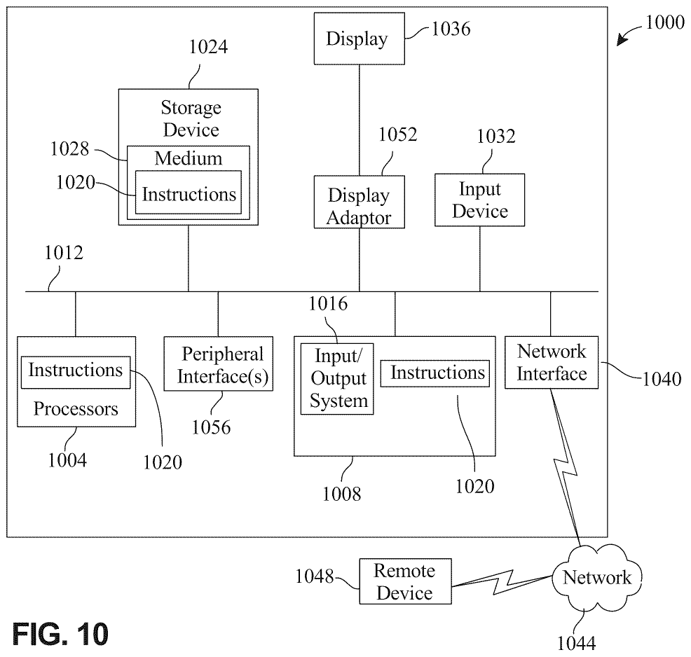

FIG. 10 shows a diagrammatic representation of one embodiment of a computing device in the exemplary form of a computer system 1000 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1000 includes a processor 1004 and a memory 1008 that communicates with each other, and with other components, via a bus 1012. Bus 1012 may include any of several types of bus structures including, but not limited to, a memory bus, a memory controller, a peripheral bus, a local bus, and any combinations thereof, using any of a variety of bus architectures.

Processor 1004 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1004 may be organized according to Von Neumann and/or Harvard architecture as a non-limiting example. Processor 1004 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), system on module (SOM), and/or system on a chip (SoC).

Memory 1008 may include various components (e.g., machine-readable media) including, but not limited to, a random-access memory component, a read only component, and any combinations thereof. In one example, a basic input/output system 1016 (BIOS), including basic routines that help to transfer information between elements within computer system 1000, such as during start-up, may be stored in memory 1008. Memory 1008 may also include (e.g., stored on one or more machine-readable media) instructions (e.g., software) 1020 embodying any one or more of the aspects and/or methodologies of the present disclosure. In another example, memory 1008 may further include any number of program modules including, but not limited to, an operating system, one or more application programs, other program modules, program data, and any combinations thereof.

Computer system 1000 may also include a storage device 1024. Examples of a storage device (e.g., storage device 1024) include, but are not limited to, a hard disk drive, a magnetic disk drive, an optical disc drive in combination with an optical medium, a solid-state memory device, and any combinations thereof. Storage device 1024 may be connected to bus 1012 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1024 (or one or more components thereof) may be removably interfaced with computer system 1000 (e.g., via an external port connector (not shown)). Particularly, storage device 1024 and an associated machine-readable medium 1028 may provide nonvolatile and/or volatile storage of machine-readable instructions, data structures, program modules, and/or other data for computer system 1000. In one example, software 1020 may reside, completely or partially, within machine-readable medium 1028. In another example, software 1020 may reside, completely or partially, within processor 1004.

Computer system 1000 may also include an input device 1032. In one example, a user of computer system 1000 may enter commands and/or other information into computer system 1000 via input device 1032. Examples of an input device 1032 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1032 may be interfaced to bus 1012 via any of a variety of interfaces (not shown) including, but not limited to, a serial interface, a parallel interface, a game port, a USB interface, a FIREWIRE interface, a direct interface to bus 1012, and any combinations thereof. Input device 1032 may include a touch screen interface that may be a part of or separate from display 1036, discussed further below. Input device 1032 may be utilized as a user selection device for selecting one or more graphical representations in a graphical interface as described above.

A user may also input commands and/or other information to computer system 1000 via storage device 1024 (e.g., a removable disk drive, a flash drive, and the like.) and/or network interface device 1040. A network interface device, such as network interface device 1040, may be utilized for connecting computer system 1000 to one or more of a variety of networks, such as network 1044, and one or more remote devices 1048 connected thereto. Examples of a network interface device include, but are not limited to, a network interface card (e.g., a mobile network interface card, an LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1044, may employ a wired and/or a wireless mode of communication. In general, any network topology may be used. Information (e.g., data, software 1020, and the like.) may be communicated to and/or from computer system 1000 via network interface device 1040.

Computer system 1000 may further include a video display adapter 1052 for communicating a displayable image to a display device, such as display 1036. Examples of a display device include, but are not limited to, a liquid crystal display (LCD), a cathode ray tube (CRT), a plasma display, a light emitting diode (LED) display, and any combinations thereof. Display adapter 1052 and display 1036 may be utilized in combination with processor 1004 to provide graphical representations of aspects of the present disclosure. In addition to a display device, computer system 1000 may include one or more other peripheral output devices including, but not limited to, an audio speaker, a printer, and any combinations thereof. Such peripheral output devices may be connected to bus 1012 via a peripheral interface 1056. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. A system for transseptal puncture site selection, comprising:
a processor; and
a memory communicatively connected to the processor, the memory containing instructions configuring the processor to:
receive a 3D organ model pertaining to an organ of a subject;
receive a treatment plan pertaining to the subject, wherein the treatment plan comprises:
at least an area of interest; and
a procedure type;
generate treatment guidance as a function of the treatment plan, wherein generating the treatment guidance comprises:
determining an optimized access route of the treatment guidance to the area of interest, wherein the optimized access route comprises at least two access routes with a corresponding numerically-rated procedure score based on at least an aggregated alignment metric and is optimized for the procedure type; and
locating a puncture site of the treatment guidance as a function of the treatment plan, wherein locating the puncture site comprises determining the puncture site using a puncture machine-learning model, wherein the puncture machine-learning model is configured to receive the treatment plan as input and output a puncture site; and
generate an overlay for the 3D organ model as a function of the puncture site and the optimized access route.

2. The system of claim 1, further comprising:
at least an ultrasound imaging device comprising at least an ultrasound sensor configured to be inserted into the subject using a catheter and capture one or more ultrasound images of an organ of the subject; and
receiving the 3D organ model comprises generating the 3D organ model as a function of the one or more ultrasound images.

3. The system of claim 2, wherein generating the 3D organ model as a function of the one or more ultrasound images comprises:
generating a set of shape parameters representing a shape of the organ as a function of the one or more ultrasound images and a shape identification model trained on a training dataset comprising historical ultrasonic images correlated with historical computed tomography scan data; and
generating the 3D organ model of the organ based on the set of shape parameters.

4. The system of claim 1, wherein the treatment plan comprises a catheter selection.

5. The system of claim 4, wherein the catheter selection comprises a pliability metric and a steerability metric.

6. The system of claim 1, wherein the puncture machine-learning model has been trained with historical data to correlate a plurality of areas of interest to a plurality of puncture sites.

7. The system of claim 1, wherein determining the optimized access route comprises:
receiving a plurality of organ constraints within the 3D organ model;
identifying a plurality of access paths as a function of the plurality of organ constraints, wherein at least one access path of the plurality of access paths comprises a portion of a boundary of at least one organ constraint of the plurality of organ constraints; and
determining the optimized access route as a function of the plurality of access paths.

8. The system of claim 1, wherein determining the optimized access route comprises:
identifying a plurality of access paths located between the puncture site and the area of interest;
calculating a path score for each access path of the plurality of access paths, wherein the path score comprises at least a catheter manipulation score; and
determining the optimized access route as a function of the catheter manipulation score.

9. The system of claim 1, wherein:
the procedure type comprises a selection of a cardiovascular device to be navigated to the area of interest; and
determining the optimized access route comprises:
identifying a plurality of access paths located between the puncture site and the area of interest;
calculating the procedure score for each access path of the plurality of access paths, wherein each access path comprises more than one or more procedure scores and wherein at least one procedure score of the more than one procedure scores is correlated to the procedure type; and
determining the optimized access route as a function of the procedure score.

10. The system of claim 9, wherein the procedure score comprises an alignment metric between the cardiovascular device and the area of interest.

11. A method for transseptal puncture site selection, the method comprising:
receiving, by at least a processor, a 3D organ model pertaining to an organ of a subject;
receiving, by the at least a processor, a treatment plan pertaining to the subject, wherein the treatment plan comprises:
at least an area of interest; and
a procedure type;
generating, by the at least a processor, treatment guidance as a function of the treatment plan, wherein generating the treatment guidance comprises:
determining an optimized access route of the treatment guidance to the area of interest, wherein the optimized access route comprises at least two access routes with a corresponding numerically-rated procedure score based on at least an aggregated alignment metric and is optimized for the procedure type; and
locating a puncture site of the treatment guidance as a function of the treatment plan, wherein locating the puncture site comprises determining the puncture site using a puncture machine-learning model, wherein the puncture machine-learning model is configured to receive the treatment plan as input and output a puncture site; and
generating, by the at least a processor, an overlay for the 3D organ model as a function of the puncture site and the optimized access route.

12. The method of claim 11, wherein:
the method further comprises receiving, by at least an ultrasound imaging device, one or more ultrasound images of the subject wherein the ultrasound imaging device comprises least an ultrasound sensor configured to be located within an esophagus of the subject and capture the one or more ultrasound images of the subject; and
receiving the 3D organ model comprises generating the 3D organ model as a function of the one or more ultrasound images.

13. The method of claim 12, wherein generating the 3D organ model as a function of the one or more ultrasound images comprises:
generating a set of shape parameters representing a shape of the organ as a function of the one or more of ultrasound images and a shape identification model trained on a training dataset comprising historical ultrasonic images correlated with historical computed tomography scan data; and
generating the 3D organ model of the organ based on the set of shape parameters.

14. The method of claim 11, wherein the treatment plan comprises a catheter selection.

15. The method of claim 14, wherein the catheter selection comprises a pliability metric and a steerability metric.

16. The method of claim 11, wherein the puncture machine-learning model has been trained with historical data to correlate a plurality of areas of interest to a plurality of puncture sites.

17. The method of claim 11, wherein determining the optimized access route comprises:
receiving a plurality of organ constraints within the 3D organ model;
identifying a plurality of access paths as a function of the plurality of organ constraints, wherein at least one access path of the plurality of access paths comprises a portion of a boundary of at least one organ constraint of the plurality of organ constraints; and
determining the optimized access route as a function of the plurality of access paths.

18. The method of claim 11, wherein determining the optimized access route comprises:
identifying a plurality of access paths located between the puncture site and the area of interest;
receiving a path score for each access path of the plurality of access paths, wherein the path score comprises at least a catheter manipulation score; and
determining the optimized access route as a function of the catheter manipulation score.

19. The method of claim 11, wherein:
the procedure type comprises a selection of a cardiovascular device to be navigated to the area of interest; and
determining the optimized access route comprises:
identifying a plurality of access paths located between the puncture site and the area of interest;
calculating the procedure score for each access path of the plurality of access paths, wherein each access path comprises more than one or more procedure scores and wherein at least one procedure score of the more than one procedure scores is correlated to the procedure type; and
determining the optimized access route as a function of the procedure score.

20. The method of claim 19, wherein the procedure score comprises an alignment metric between the cardiovascular device and the area of interest.
